(19) United States
(12) Patent Application Publication
(10) Pub. No.: US 2026/0265298 A1
(43) Pub. Date: Sep. 10, 2026

(54) PEPTIDE SUBSTRATES AND INHIBITORS OF CATHEPSIN B

(71) Applicant: The Regents of the University of California, Oakland, CA (US)

(72) Inventors: Vivian Hook, La Jolla, CA (US); Anthony O'Donoghue, San Diego, CA (US); Michael Yoon, San Diego, CA (US)

(21) Appl. No.: 19/167,300

(22) PCT Filed: Mar. 21, 2024
(86) PCT No.: PCT/US2024/020965

§ 371 Date: Sep. 19, 2025

Related U.S. Application Data
(60) Provisional application No. 63/491,460, filed on Mar. 21, 2023.

(51) Int. Cl. C07K 5/083; A61K 38/00; C12Q 1/37

(52) U.S. Cl. CPC C07K 5/0808; C12Q 1/37; A61K 38/00; G01N 2333/8139; G01N 2333/96466

(57) ABSTRACT

Pharmaceutical compositions comprising a peptidic inhibitor of cathepsin B. Methods for inhibiting neutral pH cathepsin B activity, comprising administering to a subject in need an effective amount of a peptidic inhibitor of cathepsin B, such a Z-Nle-Lys-Arg-AMC. Methods for treatment or prevention of a disease comprising administering to a subject in need an effective amount of a peptidic inhibitor of cathepsin B. Methods of diagnostic observation, research tools and methods of use in drug development are also included.

### CROSS REFERENCE TO RELATED APPLICATIONS

This application claims the priority benefit of U.S. Provisional Application No. 63/491,460, filed Mar. 21, 2023, which application is incorporated herein by reference.

### GOVERNMENT SPONSORSHIP

This invention was made with government support under grant R01NS109075 awarded by the National Institutes of Health. The government has certain rights in the invention.

### TECHNICAL FIELD

The present invention relates to inhibitors of cathepsin B for disease treatment and monitoring.

### BACKGROUND

Cathepsin B is a lysosomal cysteine protease that participates in protein degradation to maintain cellular balance of functional protein pathways (1-3). Cathepsin B belongs to the family of cysteine cathepsin proteases that together function in lysosomal protein homeostasis (4, 5). Cathepsin B and cysteine cathepsin family members normally function within lysosomes at acidic pH 4.6 (6-8). However, in numerous human diseases of neurological disorders (9-20) combined with infectious diseases, inflammation, and related conditions (21-26), cathepsin B leaks out of the lysosome into the cytosol of neutral pH 7.2 (27, 28). Cathepsin B retains its enzymatic activity at cytosolic neutral pH and activates cell death (29-32) and inflammatory (33-36) pathways that result in disease pathogenesis. In addition to the significant cellular function of cathepsin B at the neutral pH of the cytosol, cathepsin B is also present at neutral pH cellular locations of the nucleus (37-39) as well as extracellular locations, especially in human diseases such as Alzheimer's disease (40-42) and cancer (43-47).

The prominent biological functions of cathepsin B at distinct acidic and neutral pH environments of tissues indicate the need to specifically monitor cathepsin B activity over a broad pH range, without detecting other cysteine cathepsins. Currently, no such substrates exist since Z-Phe-Arg-AMC that is routinely used to assay cathepsin B in the field also monitors cathepsin L and other cysteine protease activities (48-51). Furthermore, Z-Arg-Arg-AMC has been found as a specific substrate for cathepsin B over other cysteine cathepsins (49, 50), but this substrate preferentially monitors the neutral pH activity of cathepsin B rather than the enzyme's acidic activity (50). Therefore, there is a need to rationally design selective peptide-AMC substrates that specifically monitor cathepsin B activity with high specific activity at both acidic and neutral pH conditions, without cleavage by other cysteine cathepsins.

### SUMMARY OF THE INVENTION

The disclosure provides peptide substrates and inhibitors of cathepsin B.

In embodiments, the invention provides a Z-Nle-Lys-Arg-AMC peptide that is a substrate of cathepsin B for high sensitivity monitoring of proteolytic activity. The Z-Nle-Lys-Arg-AMC substrate is superior compared to others in the field because it (1) monitors substantially higher catalytic efficiency for cathepsin B activity compared to other substrates known in the field such as Z-Arg-Arg-AMC, Z-Phe-Arg-AMC, and others, (2) monitors cathepsin B proteolytic activity over a broad pH range from acidic to neutral pH conditions, which covers the enzyme's normal acidic function in lysosomes and its neutral pH functions in diseases, and (3) specifically monitors cathepsin B and not other cysteine cathepsin proteases.

The Z-Nle-Lys-Arg-AMC substrate provides a more robust and sensitive assay for cathepsin B activity at acidic to neutral pH conditions that represent the different biological environments of cathepsin B located in lysosomes of acidic pH and in neutral pH environments of the cytosol, nuclei, and extracellular locations.

The Z-Nle-Lys-Arg-AMC is a superior substrate over other commercially used substrates for cathepsin B assay kits. Development of the Z-Nle-Lys-Arg-AMC as a cathepsin B assay kit has commercial potential for its greater sensitivity and specificity to measure cathepsin B proteolytic activity. Cathepsin B assay kits that are commercially available to researchers typically use a variant form of ZArg-Arg-AMC which this invention finds to monitor low cathepsin B activity compared to the superior ZNle-Lys-Arg-AMC substrate, the subject of this invention disclosure.

New uses for the novel Z-Nle-Lys-Arg-AMC substrate to measure cathepsin B activity include (1) superior cathepsin B assay kits as a research tool kit or reagent, (2) clinical biomarker assays of cathepsin B activity in disease conditions, using plasma and CSF patient biofluids for biomarker evaluation, and (3) novel cathepsin B assays for mammalian and other species including application to veterinary animal conditions.

In embodiments, the invention provides variant forms of Z-Nle-Lys-Arg-AMC as substrates for cathepsin B. Variant chemical forms of Z-Nle-Lys-Arg-AMC can be synthesized and utilized as novel substrates of cathepsin B. Variants of this substrate can be generated by substituting the N-terminal ‘Z’ blocking group with other groups, and substituting the C-terminal AMC fluorophore group with related groups as described herein. New uses for variant forms of Z-Nle-Lys-Arg-AMC substrates for cathepsin B assays are as described herein for the non-variant form.

In embodiments, fluorogenic substrates can monitor cathepsin B activity in live cells. The Z-Nle-Lys-Arg-AMC can be modified as Z-nKR derivatives of cresyl violet and other related fluorophores to generate a probe to monitor cathepsin B activity in live cells.

In embodiments, the fluorogenic substrates described herein can be used for (1) research tools and reagents. (2) imaging of pathogenic cathepsin B in diseases such as cancer and others involving dysregulation of cathepsin B activity, and (3) clinical treatment and biomarker evaluation of cathepsin B.

In embodiments, the activity-based probe modification of Z-Nle-Lys-Arg-AMC can be used to monitor cathepsin B activity in cells and biological samples. The tripeptide Nle-Lys-Arg can be covalently linked to a reporter (such as fluorescent reporter Cy5 and others, or biotin and others) and quencher group (such as sulfo-QSY21) to generate a novel activity based probe (ABP) of cathepsin B. Firstly, the ABP probe is useful for live cell imaging of cathepsin B whereby cathepsin B cleaves the peptide substrate which releases the quencher and resulting fluorescence indicates proteolytic activity. Secondly, the ABP probe is useful for biochemical labeling of cathepsin B in biological samples since the ABP becomes covalently linked to the protease enzyme.

In embodiments, the peptide probes of cathepsin B provide novel research tools and reagents for monitoring cathepsin B activity dysregulation in human disease tissues such as cancer.

In embodiments, the cathepsin B substrates described herein, such as Z-Nle-Lys-Arg-AMC, can be used as peptide-based inhibitors of cathepsin B. For example, warhead modification of Z-Nle-Lys-Arg-AMC can generate novel, effective inhibitors of cathepsin B. The C-terminal warheads of AOMK, VS, and other active pharmaceutical ingredients and therapeutic agents (“warheads”) can replace the AMC group of Z-Nle-Lys-Arg-AMC to generate Z-Nle-Lys-Arg-warhead as novel inhibitors of cathepsin B.

The novel cathepsin B inhibitors have significant application in drug development and treatment for numerous human diseases such as: (a) brain disorders including Alzheimer's disease and related neurodegenerative diseases, traumatic brain injury, stroke, and others, (b) cancers, (c) infectious diseases, (d) inflammatory diseases and others. Various treatable human diseases and conditions that involve cathepsin B are described below.

Brain disorders include for example ALS, Alzheimer's disease, Alzheimer's periodontitis, cerebral aneurysm, chronic opiate use, Huntington's disease, inflammatory pain, ischemia and stroke, meningitis, multiple sclerosis, Parkinson's disease. Pick's disease, spinal cord contusion, traumatic brain injury, neurodegeneration, and neuroinflammation.

Other human diseases include for example autoinflammatory disease, atherosclerosis, cancer and metastasis, lung disease, pancreatitis, rheumatoid arthritis, viral infections, and TNF-alpha induced hepatic apoptotic cell death.

In embodiments, the Nle-Lys-Arg substrate can be used as a linker for antibody-drug conjugates for cathepsin B cleavage to release drugs at pathogenic tissue locations in diseases such as cancer and others. The tripeptide Nle-Lys-Arg can be used as a linker in drug-conjugate complexes whereby the novel tripeptide Nle-Lys-Arg is efficiently cleaved by cathepsin B to release the drug from the conjugate. The released drug will be located at the pathogenic cathepsin B site to combat disease conditions such as cancer, and related diseases and conditions.

In summary, embodiments of this invention of the novel Z-Nle-Lys-Arg-AMC provide superior substrate of cathepsin B and variant modifications for new substrates, inhibitors, and drug conjugates have applications in high sensitivity assay of cathepsin B to monitor proteolytic activity across broad acid to neutral pH conditions with specificity for cathepsin B over other cysteine cathepsin proteases, novel chemical activity probes of cathepsin B for research, clinical biomarker assays of cathepsin B in diseases for diagnostics evaluation, and pharmaceutical inhibitors of cathepsin B.

### BRIEF DESCRIPTION OF THE DRAWINGS

FIGS. 1a-1f. Exopeptidase and endopeptidase cleavages by cathepsin B compared to other cysteine cathepsins assessed by MSP-MS. The proteolytic cleavage properties of cathepsin B and the cysteine cathepsins K, L, S, V, and X were assessed at pH 4.6 and pH 7.2 by substrate profiling with the peptide library in MSP-MS assays. MSP-MS analyses were conducted for cathepsins B, K, L, S, V, and X as shown in FIGS. 1a-1f, respectively. After incubation of each protease with the peptide library for 15 minutes, with the exception that cathepsin X was incubated with the library for 60 min, cleavage products were identified by nano-LC-MS/MS as described in the methods. The number of cleavages at each peptide bond, number 1 to 13, is illustrated for the cysteine cathepsins B, K, L, S, V, and X.

FIGS. 2a-2b. Assessment of distinct cathepsin B preferred residues at P1 to P3 positions adjacent to P1-P1′ cleavage sites compared to other cysteine cathepsins. The preferred residues of human cathepsin B at P1 to P3 positions were compared to that of cathepsins K, L, S, and V at pH 4.6 (FIG. 2a) and pH 7.2 (FIG. 2b) shown in heat maps of z-scores. Preferred residues are indicated by positive scores (green shades) and non-preferred residues are indicated by negative scores (yellow shades). The residues circled in red indicate the unique residues preferred by cathepsin B over the other cathepsins at each of the P1 to P3 positions.

FIG. 3. Z-Nle-Lys-Arg-AMC monitors high cathepsin B specific activity at pH 4.6 and pH 7.2 compared to related tripeptide and dipeptide substrates Z-Nle-Lys-Arg-AMC (Z-n-K-R-AMC) and the indicated tripeptide and dipeptide fluorogenic substrates (at 4 μM) were compared for proteolysis by cathepsin B at pH 4.6 (blue shade bars) and pH 7.2 (red shade bars). One letter codes for amino acids are shown for the peptidic substrates. Proteolytic activity of cathepsin B is shown as the mean+SD (n=4).

FIGS. 4a-4b. Cathepsin B cleaves Z-Nle-Lys-Arg-AMC over a broad pH range. (FIG. 4a) pH profile of human cathepsin B activity with Z-Nle-Lys-Arg-AMC compared to Z-Arg-Arg-AMC and Z-Phe-Arg-AMC substrates. (i) Cathepsin B pH profiles with the three different substrates (4 μM) were assessed at pH 2 to 9. Data points are shown as the mean±SD (n=3). (ii) Structure of Z-Nle-Lys-Arg-AMC is shown. This substrate provides the highest specific activity for cathepsin B activity. (FIG. 4b) Kinetic kcat/Km values for cathepsin B activity with the substrates Z-Nle-Lys-Arg, Z-Arg-Arg-AMC and Z-Phe-Arg-AMC. kcat/Km values for each of the substrates at pH 4.6 and pH 7.2 were calculated for cathepsin B by plotting the rate of AMC product formation of the v-axis with its respective substrate concentration of the x-axis as described in the methods.

FIGS. 5a-5c. Z-Nle-Lys-Arg-AMC displays specificity for cathepsin B over other cysteine cathepsins L, K, S, and V. (FIG. 5a) Z-Nle-Lys-Arg-AMC. Cathepsin B specific activity for cleavage of Z-Nle-Lys-Arg-AMC was compared to that of cathepsins L, K, S. and V at pH 7.2, 5.5, and 4.6. (FIG. 5b) Z-Arg-Arg-AMC. Cathepsin B specific activity for cleavage of Z-Arg-Arg-AMC was compared to that of cathepsins L, K, S, and V at pH 7.2, 5.5, and 4.6. (FIG. 5c) Z-Phe-Arg-AMC. Cathepsin B specific activity for cleavage of Z-Phe-Arg-AMC was compared to that of cathepsins L, K, S, and V at pH 7.2, 5.5, and 4.6. All data are shown as the mean+SD (n=3).

FIGS. 6a-6c. Cathepsin B assessed as CA-074-sensitive activity in neuroblastoma and microglia cells assessed with Z-Nle-Lys-Arg-AMC compared to Z-Arg-Arg-AMC and Z-Phe-Arg-AMC. Proteolytic activity of cathepsin B and other cysteine cathepsins were measured by fluorogenic assays of cell homogenates incubated with Z-Nle-Lys-Arg-AMC, Z-Arg-Arg-AMC, and Z-Phe-Arg-AMC substrates (60 μM) at pH 4.6, 5.5 and 7.2 with or without pre-incubation with CA-074, a potent and selective cathepsin B inhibitor. Cells assessed consisted of (FIG. 6a) human neuroblastoma SHSY-5Y, (FIG. 6b) human neuroblastoma SK-N-MC, and (FIG. 6c) mouse microglia BV2. Results display specific activity in the absence and presence of CA-074 which indicates cathepsin B specific activity. Data show cathepsin B activity as pmol AMC/μg enzyme protein (30 min incubation) as the mean+SD (n=3).

FIGS. 7a-7f Cathepsins B, K, L, S, and V cleavage profiling analyzed by MSP-MS. Volcano plots of cathepsin B (FIG. 7a), cathepsin K (FIG. 7b), cathepsin L (FIG. 7c), cathepsin S (FIG. 7d), cathepsin V (FIG. 7e), and cathepsin X (FIG. 7f) peptide cleavages from MSP-MS data show the log 2 ratios of relative quantities of peptide products generated by each of these cathepsins compared to no enzyme activity controls, illustrated by −log 10 p values. Peptide products generated with at least an 8-fold change above controls and with p<0.05 were analyzed for the frequencies of amino acid residues at the P4-P4′ positions of the P1-↓P1′ cleavage site in the heatmaps and iceLogos depicted in FIGS. 2 and 3.

FIGS. 8a-8b. Cathepsin B and cathepsin K cleavage profiles at pH 4.6 and pH 7.2 assessed for preferred residues at P4 to P4′ positions adjacent to P1-P1′ cleavage sites. (FIG. 8a) Cathepsin B cleavage profiles at pH 4.6 and pH 7.2. The preference of cathepsin B cleavages for amino acid residues at each of the P4 to P4′ positions adjacent to the P1-P1′ cleavage site was deduced from MSP-MS data cleavage profiling data. Z-scores provided quantitation of preferred (green shades) and non-preferred (yellow shades) residues illustrated in a heat map. The z-scores were utilized for iceLogo illustration of the primary preferred residues at the P4 to P4′ positions shown above the mid-line, with non-preferred residues shown below the mid-line. The purple letters indicate significance of p<0.05, and the black letters indicate p<0.3. (FIG. 8b) Cathepsin K cleavage profiles at pH 4.6 and pH 7.2. The preference of cathepsin K cleavages for residues at P4 to P4′ positions was defined from MSP-MS data, indicated by z-scores shown in a heat map for preferred residues (green shades) and non-preferred residues (yellow shades). The primary preferred residues, based on z-scores, were plotted in iceLogo illustrations. The purple letters indicate significance of p<0.05, and the black letters indicate p<0.3.

FIGS. 9a-9c. Cathepsins L, S, and V cleavage profiles at pH 4.6 and pH 7.2 assessed for preferred residues at P4 to P4′ positions. The preferences of each cathepsin protease for residues at P4 to P4′ positions were defined by MSP-MS data, and indicated z-scores for preferred residues (green shades) and non-preferred residues (yellow shades). IceLogo illustrated the main preferred residues (above the line) and preferred residues with significance of p<0.05 are shown in purple letters.

FIGS. 10a-10c. Specific cathepsin B activity monitored with novel Z-Nle-Lys-Arg-AMC substrate in human neuroblastoma and mouse microglia cells. Cathepsin B activity in cell homogenates from human neuroblastoma SHSY-5Y cells (FIG. 10a), human neuroblastoma SKN-MC cells (FIG. 10b), and mouse microglia BV2 cells (FIG. 10c) was assessed with the substrates Z-Nle-Lys-Arg-AMC, Z-Arg-Arg-AMC, and Z-Phe-Arg-AMC (60 μM) at pH 4.6, 5.5, and 7.2. Data show cathepsin B activity as pmol AMC/g enzyme protein (30 min incubation) as the mean+SD (n=3). Significance is indicated by *p<0.05 (student's t-test) comparing proteolytic activity with the of substrates Z-Arg-Arg-AMC and Z-Phe-Arg-AMC compared to Z-Nle-Lys-Arg-AMC.

FIGS. 11a-11b. Mouse cathepsin B displays pH-dependent activity with the substrates Z-Nle-Arg-Lys-AMC, Z-Arg-Arg-AMC, and Z-Phe-Arg-AMC. (FIG. 11a) pH-dependence of mouse cathepsin B activity with Z-Nle-Lys-Arg-AMC compared to Z-Arg-Arg-AMC and Z-Phe-Arg-AMC substrates. Cathepsin B specific activity with the three different substrates (40 μM) were assessed at pH 4.6, 5.5, and 7.2. Data points are shown as the mean+SD (n=3). (FIG. 11b) Kinetic kcat/Km values for cathepsin B activity with the substrates Z-Nle-Lys-Arg, Z-Arg-Arg-AMC and Z-Phe-Arg-AMC. kcat/Km values for each of the substrate(s at pH 4.6 and pH 7.2 were calculated for mouse cathepsin B as described in the methods. (The asterisk indicates that the kcat/Km was determined from the linear slope of the curve since the curve did not plateau out.)

FIGS. 12a-12b. Regulation of cathepsin B activity during cell death induced by low nutrient stress in microglia cells. FIG. 12a shows cell death in microglia cells induced by low nutrient stress. Microglia cells (mouse BV2 cells) were treated with low nutrient culture media (0% fetal bovine serum, FBS) compared to a normal nutrient condition (10% FSB) for 24 hours. The low nutrient stress condition induced cell death monitored by LDH (lactate dehydrogenase) released into the culture media. The stress-induced increase in cell death was reduced by the inhibitor Z-Arg-Lys-AOMK (2 μM), a selective inhibitor of cathepsin B. (n=6, mean+sem, *p<0.05, student's t-test). FIG. 12b shows cathepsin B activity is increased in the cytosol of microglia cells during low nutrient stress. Cathepsin B activity was elevated in cytosol isolated from microglia cells after treatment with low nutrient stress (0% FBS) for 24 hours. Cathepsin B activity was measured with the specific substrate Z-Nle-Lys-Arg-AMC (60 mM in assay). Furthermore, the increase in cytosolic cathepsin B activity was blocked by the cathepsin B inhibitor Z-Arg-Lys-AOMK (2 μM) during low nutrient stress. (n=6, mean+sem, *p<0.05, student's t-test).

FIGS. 13a-13e. Ttraumatic brain injury (TBI) in mice results in elevated brain cathepsin B activity, involved in motor deficit. FIG. 13a shows TBI induced motor deficit in mice subjected to the controlled cortical impact (CCI) model of TBI. Mice were subjected to the controlled cortical impact (CCI) model of TBI. Motor function was assessed by the rotarod assay on days 1, 3, and 7 after TBI. Control sham mice received surgery of the scalp but no TBI. Rotarod data are shown as mean+SD (n=5/group, mean+sem, *p<0.05, student's t-test). FIG. 13b shows brain lesion induced by TBI. The TBI injury was conducted on the ipsilateral side of the brain cortex, shown by the tissue lesion at the site of TBI impact. FIG. 13c shows elevated cathepsin B activity in homogenate of mouse ipsilateral brain cortex after TBI. Cathepsin B activity in homogenate of mouse brain cortex was measured with the Z-Nle-Lys-Arg-AMC substrate on day 6 after TBI. These TBI mice were treated with vehicle or Z-Arg-Lys-AOMK inhibitor (20 mg/kg, ip). (n=6 per group for vehicle-treated, and n=4 per group for treatment with inhibitor Z-Arg-Lys-AOMK; mean+sem, p<0.05, student's t-test). FIG. 13d shows elevation of cytosolic cathepsin B activity in mouse ipsilateral brain cortex after TBI. Cytosol from brain homogenate (obtained on day 6 after TBI) was isolated by differential density centrifugation. Cathepsin B in the cytosol from brain cortex was measured by the substrate Z-Nle-Lys-Arg-AMC substrate. (n=6 per group for vehicle treated, and n=4 per group for treatment with inhibitor Z-Arg-Lys-AOMK; mean+sem, p<0.05, student's t-test). FIG. 13e shows inhibitor of cathepsin B improves motor deficit induced by TBI. TBI mice were treated with Z-Arg-Lys-AOMK inhibitor (20 mg/kg, ip) one day before TBI, on the day of TBI, and on day 1 after TBI. Motor function was measured on day 1 after TBI. (n=6 per group for vehicle-treated, and n=4 per group for treatment with inhibitor Z-Arg-Lys-AOMK; mean+sem, p<0.05, student's t-test).

### DETAILED DESCRIPTION

In embodiments, the invention provides peptidic inhibitors of cathepsin B, and methods of use and preparation thereof, comprising a tri-peptide Nle-Lys-Arg.

In embodiments, the invention provides a peptidic inhibitor of cathepsin B comprising a carboxy-terminally capped and/or an amino-terminally capped tri-peptide Nle-Lys-Arg. In embodiments, the invention provides a peptidic inhibitor of cathepsin B, wherein the inhibitor is carboxy-terminally capped with AMC. In embodiments, the invention provides a peptidic inhibitor of cathepsin B, wherein the amino-terminal cap is a benzyloxycarbonyl (Z). In embodiments, the invention provides a peptidic inhibitor of cathepsin B comprising Z-Nle-Lys-Arg-AMC.

In embodiments, the invention provides a pharmaceutical or diagnostic composition comprising the peptidic inhibitor of cathepsin B as described herein and a pharmaceutically acceptable excipient.

In embodiments, the invention provides a method of inhibiting neutral pH cathepsin B activity, comprising administering to a mammalian subject in need an effective amount of a peptidic inhibitor of cathepsin B as described herein.

In embodiments, the invention provides a method for treatment of a disease or disorder comprising administering to a mammalian subject in need an effective amount of a pharmaceutical composition comprising a peptidic inhibitor of cathepsin B as described herein. In embodiments, the invention provides that the disease or disorder is neurological. In embodiments, the invention provides that the disease is autoinflammatory disease, atherosclerosis, cancer and metastasis, lung disease, pancreatitis, rheumatoid arthritis, viral infections, or TNF-alpha induced hepatic apoptotic cell death.

In embodiments, the invention provides a method for detecting cathepsin B activity in a composition of interest comprising administering to the composition an effective amount of a composition comprising a peptidic inhibitor of cathepsin B as described herein, and detecting cathepsin B activity.

In embodiments, the invention provides that the carboxy-terminal cap is AMC. In embodiments, the invention provides that the carboxy-terminal cap is alternatively selected from another electrophilic warhead that irreversibly reacts with the active site of cysteine proteases, including epoxysuccinates, vinyl esters, vinyl sulfones, allyl sulfones, vinyl sulfonates, hydroxymethylketone, diazomethylketones, aryloxymethylketones, sulfonium methvlketones, fluoromethylketones or chloromethylketones. In addition, the invention provides that the cysteine protease inhibitor is selected from a reversible cysteine reactive warhead that includes aldehydes, thiomethylketones, oxymethylketones, cyclic ketones, amidomethylketones, nitriles, or various 1.2-dicarbonyl motifs.

In embodiments, the invention provides that the peptidic inhibitor of cathepsin B has an amino-terminal cap that is a benzyloxycarbonyl (Z). In embodiments, the amino-terminal cap is alternatively an acetyl (Ac), benzoyl (Bz), benzyl (Bzl), tert-butyloxycarbonyl (Boc), pyrazinylcarbonyl, cinnamoyl, naphthalene (Nap), fluorenylmethoxycarbonyl (Fmoc), pyrene (Pyr), phenothiazine (PTZ), morpholinyl, trifluoroacetamide, tosyl or other N-terminal protecting groups.

In embodiments, the invention provides that the peptidic inhibitor of cathepsin B has a formula Z-Nle-Lys-Arg-AMC.

In embodiments, the invention provides pharmaceutical compositions for treatment or diagnostic observation of a disease or condition comprising the peptidic inhibitor of cathepsin B as described herein and a pharmaceutically acceptable excipient.

In embodiments, the disease or condition is neurological. In embodiments, the neurological disease or condition is Alzheimer's disease, traumatic brain injury (TBI), neurodegenerative conditions, TBI and ischemia, Parkinson's disease or another neurodegenerative condition or behavioral deficit.

In embodiments, the disease or condition is inflammatory, infectious or metabolic. In embodiments, the inflammatory or infectious disease or condition is atherosclerosis, mucopolysaccaridosis, pancreatitis, Mycobacterium tuberculosis, dengue virus, pancreatitis, pneumoccal meningitis, rheumatoid arthritis, or HIV.

In embodiments, the disease or condition is metabolic, such as Niemann-Pick disease or lysosomal storage disorders.

In embodiments, the disease is cancer. In embodiments, the cancer is colorectal cancer or breast cancer (due to extracellular cathepsin B) or thyroid cancer (due to nuclear cathepsin B).

In embodiments, the condition is characterized by cytosolic cathepsin B initiated apoptotic cell death or activation of inflammatory IL-1beta production.

In embodiments, the disease or condition occurs at a neutral pH site in the subject.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.): Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone. C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein the term “pharmaceutical composition” refers to pharmaceutically acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which the active compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens: antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective amount” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset: or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment.” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent.

As used herein, and unless otherwise specified, the term “subject” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

As used herein, and unless otherwise specified, a compound described herein is intended to encompass all possible stereoisomers, unless a particular stereochemistry is specified. Where structural isomers of a compound are interconvertible via a low energy barrier, the compound may exist as a single tautomer or a mixture of tautomers. This can take the form of proton tautomerism; or so-called valence tautomerism in the compound, e.g., that contain an aromatic moiety.

### EXAMPLES

The biological and pathological roles of cathepsin B occur in acidic lysosomes and at the neutral pH of cytosol, nuclei, and extracellular locations. Importantly, cathepsin B displays different substrate cleavage properties at acidic compared to neutral pH conditions. It is, therefore, desirable to develop specific substrates for cathepsin B that measure its activity over broad pH ranges. Current substrates used to monitor cathepsin B activity consist of Z-Phe-Arg-AMC and Z-Arg-Arg-AMC, but they lack specificity since they are cleaved by other cysteine cathepsins. Furthermore, Z-Arg-Arg-AMC monitors cathepsin B activity at neutral pH and displays minimal activity at acidic pH. Therefore the purpose of this study was to design and validate specific fluorogenic peptide substrates that can monitor cathepsin B activity over a broad pH range from acidic to neutral pH conditions. In-depth cleavage properties of cathepsin B were compared to that of the cysteine cathepsins K, L, S, V, and X by multiplex substrate profiling mass spectrometry (MSP-MS) at pH 4.6 and pH 7.2. Analysis of the preferred and non-preferred residues at the P3, P2, and P1 positions adjacent to the P1-P1′ cleavage sites predicted the tripeptide Z-Nle-Lys-Arg-AMC as a preferred substrate for cathepsin B. Significantly, Z-Nle-Lys-Arg-AMC displayed the advantageous properties of measuring high cathepsin B specific activity over acidic to neutral pHs and was specifically cleaved by cathepsin B over the other cysteine cathepsins. Z-Nle-Lys-Arg-AMC specifically measured cathepsin B activity in neuronal and glia cells which were consistent with relative abundances of cathepsin B protein. These findings validate Z-Nle-Lys-Arg-AMC as a novel substrate that specifically monitors cathepsin B activity over a broad pH range.

The strategy of this study was to design specific substrates for cathepsin B based on its unique pH-dependent cleavage properties at acidic pH 4.6 compared to neutral pH 7.2. Cleavage properties of cathepsin B were determined by multiplex substrate profiling mass spectrometry (MSP-MS) that utilizes a defined peptide library designed to contain all known amino acids adjacent to protease cleavage sites (51). These substrate properties of cathepsin B were compared to that of the other cysteine cathepsins L, K, S, V. and X at acidic and neutral pH conditions. The unique cleavage properties of cathepsin B allowed design of Z-peptide-AMC substrates for assessment of broad pH monitoring of cathepsin B activity, with specificity shown by a lack of cleavage by other cysteine cathepsins.

Results identified Z-Nle-Lys-Arg-AMC that monitors high specific activity of cathepsin B at acidic to neutral pHs, and this substrate is specific for cathepsin B over other cysteine cathepsins L, K, S, and V. Kinetic studies demonstrated greater catalytic efficiency of Z-Nle-Lys-Arg-AMC for analysis of acidic and neutral pH cathepsin B activity compared to the Z-Arg-Arg-AMC substrate, currently used in the field as a specific cathepsin B substrate (49, 50). Furthermore, the novel Z-Nle-Lys-Arg-AMC substrate was demonstrated to specifically measure cathepsin B activity in neuronal and glia cells that contain varying levels of cathepsin B and other cathepsins (determined by proteomics assessment). Overall, the novel Z-Nle-Lys-Arg-AMC substrate advantageously monitors specific cathepsin B activity over a broad pH range of acidic to neutral pH conditions and, thus, will be useful for assessment of cathepsin B activity at physiological pH conditions.

### Results

Analysis of cathepsin B substrates Z-Phe-Arg-AMC and Z-Arg-Arg-AMC for cleavage by cysteine cathepsins demonstrate lack of specificity. The specific activity of human cathepsin B was compared with the fluorogenic substrates Z-Phe-Arg-AMC and Z-Arg-Arg-AMC with that of other cysteine cathepsins (Table 1). Both of these two substrates were cleaved by cathepsin B and other cysteine cathepsins L, K, S, and V, indicating that these substrates are not specific for cathepsin B. These assays also demonstrated that cathepsins B, K, and S were active at both acidic and neutral pHs of 4.6 and 7.2, respectively, but cathepsins L and V displayed activity only at acidic pH 4.6 and not pH 7.2. These findings indicate the lack of specificity of Z-Phe-Arg-AMC and ZArg-Arg-AMC substrates for cathepsin B.

TABLE 1
Example of variant substrates of Z-Nle-Lys-Arg-AMC
with different N-blocking groups and C-groups

| N-blocking group | C-group |
|---|---|
| Z, carboxybenzyl | 7-amino-4-methylcoumarin (AMC) |
| Ac | 7-amino-4-carbamoylmethylcoumarin (ACC) |
| Boc | para-nitroaniline (pNA) |
| Bz | Rhodamine 110 |
| Glt | |
| Suc | |

Comparison of Z-Arg-Arg-AMC cleavage by cathepsin B at acidic lysosomal pH 4.6 compared to neutral pH 7.2 of the cytosol showed that this substrate displays pH-dependent cleavage properties. Z-Arg-Arg-AMC is readily cleaved by cathepsin B at neutral pH, but this substrate has poor cleavage activity at acidic pH that is one-third of the specific activity at neutral pH.

The lack of specificity of Z-Phe-Arg-AMC and Z-Arg-Arg-AMC for cathepsin B compared to other cysteine cathepsins, and preference of Z-Arg-Arg-AMC to monitor neutral pH cathepsin B, indicates the need for development of specific substrates of cathepsin B operating over a broad pH range.

Cathepsin B displays distinct cleavage properties compared to other cysteine cathepsins revealed by multiplex substrate profiling mass spectrometry (MSP-MS). The substrate cleavage properties of human cathepsin B were compared to that of the cysteine cathepsins K, L, V, S, and X by MSPMS analysis. MSP-MS was conducted by incubation of each protease with a library of 228 14-mer peptides at acidic pH 4.6 and neutral pH 7.2, and cleavage products were identified and analyzed by LC-MS/MS tandem mass spectrometry. The peptide library used for MSP-MS analysis was designed to contain all known protease cleavage sites with respect to amino acid residues at cleavage sites combined with neighboring residues. These cysteine proteases cleaved peptides at both pH 4.6 and pH 7.2 in the MSP-MS study, with the exception of cathepsin X that generated a low number of cleavage products at only pH 4.6 (shown by volcano plots in FIGS. 7a-7f).

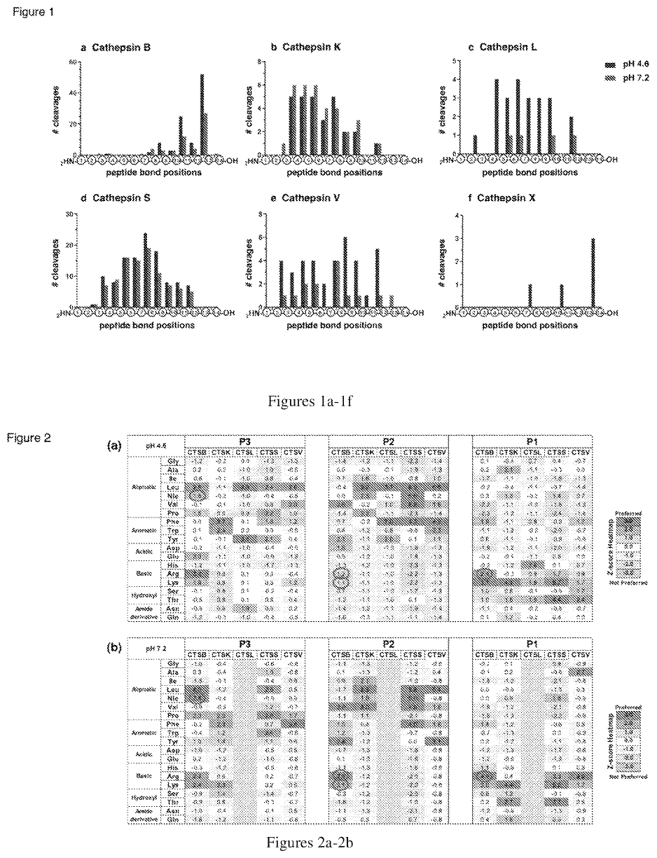

The MSP-MS analysis of cleavage site locations within the 14-mer peptides showed that cathepsin B differs in its exopeptidase and endopeptidase cleavages compared to the other cysteine cathepsins. Cathepsin B displayed major dipeptidyl carboxypeptidase (DPCP) cleavages occurring between residues 12 and 13 of the peptide substrates, as well as lower endopeptidase cleavages occurring within the 14-mer substrates (FIG. 1a). These DPCP and endopeptidase activities of cathepsin B distinguish it from the cathepsins K, L, S, and V which displayed endopeptidase activities and no carboxypeptidase activity (FIGS. 1b-e). Cathepsin X displayed endopeptidase and monopeptidyl carboxypeptidase cleavage (with a low number of 5 peptides cleaved) which differs from cathepsin B (FIG. 1f).

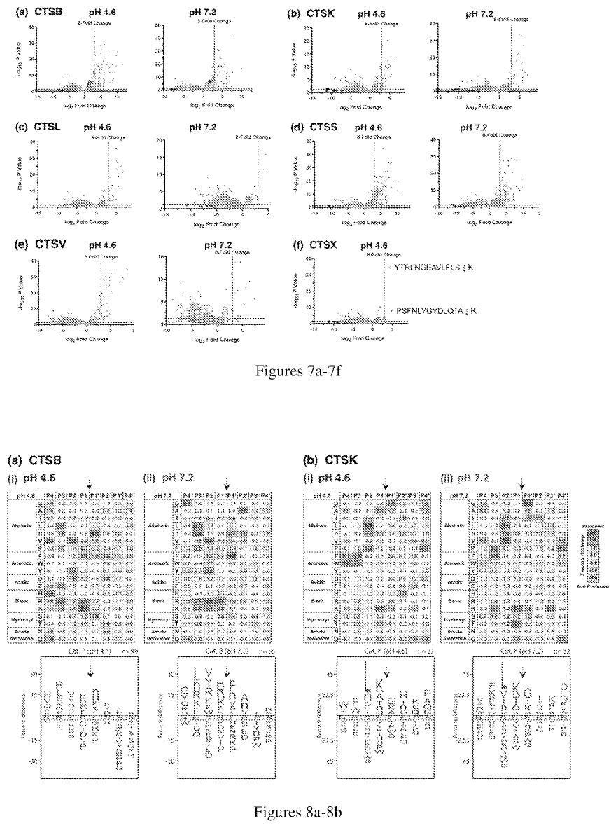

The substrate cleavage specificity of cathepsin B was compared to that of the other cysteine cathepsins by assessing preferred residues at the P4-P4′ positions surrounding the P1-P1′ cleavage site using z-scores and iceLogo (FIGS. 8a-8b, 9a-9c). Heat maps of z-scores illustrated the preferred residues (green shade) and non-preferred residues (yellow shade) at each of the P4 to P4′ residues. All amino acids were assessed with the exception of methionine and cysteine; norleucine (n) was used as a sulfur-free isostere of methionine.

Comparison of the heat maps at pH 4.6 and pH 7.2 for cathepsins B, K, S, and V showed differing patterns for each of these proteases at the acidic compared to neutral pH conditions (FIGS. 8a-8b, 9a-9c). The iceLogo format illustrates the main preferred residues at P4 to P4′ positions. We focused on noting the preferred residues at positions P3 to P1 at pH 4.6 and pH 7.2 that can be utilized for design of Z-peptide-AMC fluorogenic substrates spanning acidic and neutral pH activities of cathepsin B (FIGS. 2a-2b).

For cathepsin B at the P3 position, the enzyme displayed preferences for norleucine (Nle) and basic residues (Arg and Lys) at the two pH conditions (FIGS. 2a-2b). At the P2 position, the enzyme preferred basic (Arg, Lys) and hydrophobic (Val) residues at pH 4.6 and pH 7.2. At the P1 position, cathepsin B showed preference for basic residues (Arg and Lys) at both pHs.

In contrast, cathepsins K, S. and V displayed different preferences for residues at P3 to P1 compared to cathepsin B (FIGS. 2a-2b). At the P3 position, Nle was not preferred by cathepsins K, S, and V at both pHs. At the P2 position, basic residues were not preferred by cathepsins K, S and V at both pHs. But hydrophobic Val at P2 was preferred by cathepsin K at pH 7.2, preferred by cathepsin S at pH 4.6 and pH 7.2, and cathepsin V showed moderate preference for Val at P2 at both pHs. With respect to the P1 position, cathepsins K and S preferred Lys but not Arg at pH 4.6 and 7.2, cathepsin L did not prefer basic residues for its activity at pH 4.6 (no activity at pH 7.2), and cathepsin V preferred Lys at both pHs and also preferred Arg at pH 7.2.

Compilation of preferred residues among cathepsins B, K, L, S, and V for positions P3 to P1 illustrated the distinct preferences of cathepsin B compared to those of cathepsins K, L, S. and V (FIGS. 2a-2b). For cathepsin B at the P3 position. Nle was indicated as being preferred at both pH 4.6 and pH 7.2, based on the positive score of 1.6 at pH 4.6 and score of 2.8 at pH 7.2. But all the other cathepsins had a negative z-score for Nle at P3. At the P2 position cleavage for cathepsin B, Lys and Arg were found to be preferred at both pH 4.6 and pH 7.2, based on the z-scores of 1.1 and 1.2 at pH 4.6 and 2.1 and 2.8 at pH 7.2, respectively, while the other cathepsins had negative scores at the two pHs. At the P1 position, cathepsin B preferred Arg that was a lesser preference by the other cathepsins K, L, S, and V. Importantly, these results highlight Nle-Lys-Arg as the preferred residues at P3-P2-P1 positions compared to that of the other cysteine cathepsins assessed. These findings suggested that the tripeptide Z-Nle-Lys-Arg-AMC or Z-Nle-Arg-Arg-AMC may be specific substrates of cathepsin B activity at both acidic and neutral pH conditions.

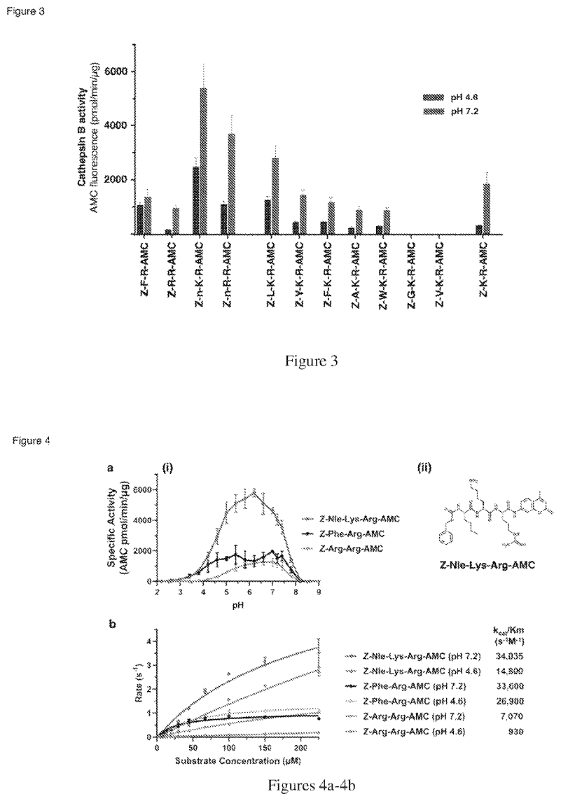

Design and validation of Z-Nle-Arg-Lys-AMC as a specific cathepsin B substrate for acidic and neutral pH conditions. We synthesized Z-Nle-Lys-Arg-AMC and Z-Nle-Arg-Arg-AMC and quantified cathepsin B activity at acidic and neutral pH conditions (FIG. 3). The specific activity of cathepsin B for cleaving Z-Nle-Lys-Arg-AMC was higher than all other substrates at both pHs. The Z-Nle-Arg-Arg-AMC substrate displayed about 30% and 50% less activity at pH 4.6 and pH 7.2, respectively, compared to Z-Nle-Lys-Arg-AMC. The Z-Nle-Lys-Arg-AMC substrate monitored higher cathepsin B specific activity compared to the commonly used cathepsin B substrates of ZPhe-Arg-AMC and Z-Arg-Arg-AMC.

Modifications of the P3 residues of variant tripeptide sequences of Z-Nle-Lys-Arg-AMC were assessed. Substitution of Leu for Nle at the P3 position generated the substrate Z-Leu-Lys-Arg-AMC, which showed less activity than Z-Nle-Lys-Arg-AMC. When the P3 residue was modified to Tyr, Phe, Ala, or Trp, the resultant substrates displayed decreased activities at both acid and neutral pHs. When the P3 residue was changed to Gly or Val, no cathepsin B activity was observed, correlating with Gly and Val having negative z-scores in the MSP-MS data.

Assessment of the dibasic substrate of Z-Lys-Arg-AMC that lacks a P3 amino acid but has the most favorable P2 and P1 residues had lower activity than Z-Nle-Lys-Arg-AMC, but higher activity than substrates that have an unfavorable amino acid at the P3 position.

Overall, analysis of Z-Nle-Lys-Arg-AMC and related peptide-AMC substrates demonstrated that the optimized residues at P3, P2, and P1 positions provided design and validation of the novel high specific activity Z-Nle-Lys-Arg-AMC substrate of cathepsin B.

Z-Nle-Lys-Arg-AMC substrate monitors cathepsin B over a broad pH range. The pH properties of Z-Nle-Lys-Arg-AMC were assessed and compared to the established substrates Z-Arg-Arg-AMC and Z-Phe-Arg-AMC (FIG. 4a). Z-Nle-Lys-Arg-AMC showed a broad pH optimum of pH 4.5 to pH 7.5 that represented 50% of the maximal activity observed at pH 5.5-6.5, which was above the activity measurements monitored of the other two Z-Arg-Arg-AMC and Z-Phe-Arg-AMC substrates. The structure of Z-Nle-Lys-Arg-AMC illustrates its side chains of the tripeptide residues of this high activity substrate (FIG. 4a). Kinetic assessment by kcat/Km values showed the high catalytic efficiency of cathepsin B activity monitored with Z-Nle-Lys-Arg-AMC and lower catalytic efficiency with Z-Arg-Arg-AMC at both pH 7.2 and pH 4.6 (FIG. 4b). Z-Phe-Arg-AMC displayed high kcat/Km values similar to Z-Nle-Lys-Arg at pH 7.2, and had higher catalytic efficiency at pH 4.6.

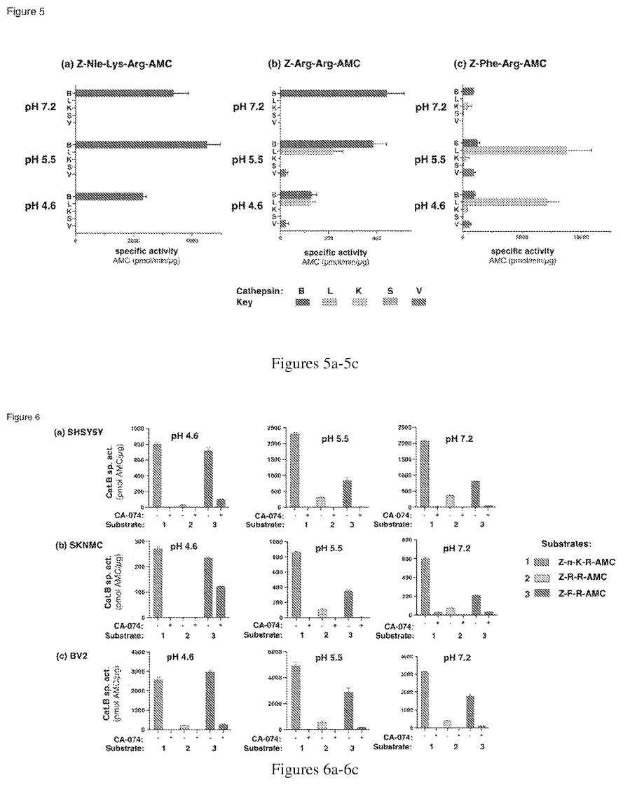

Z-Nle-Lys-Arg-AMC specifically monitors cathepsin B activity compared to other cysteine cathepsins. Z-Nle-Lys-Arg-AMC specifically detected cathepsin B activity, and not cathepsins L. K. S, and V activities evaluated at pH 4.6, 5.5, and 7.2 (FIGS. 5a-5c). In contrast, Z-Arg-Arg-AMC was not specific for cathepsin B since this substrate was cleaved by cathepsins L and V. The Z-Phe-Arg-AMC substrate was cleaved by cathepsin L with greater activity than that for cathepsin B; this substrate was also cleaved by cathepsins K and V. These data demonstrate Z-Nle-Lys-Arg-AMC as a specific substrate for cathepsin B.

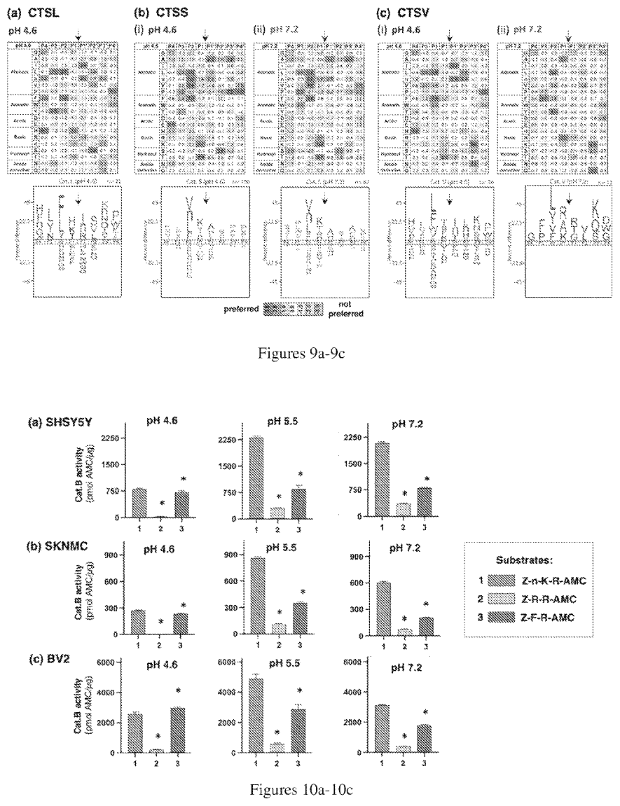

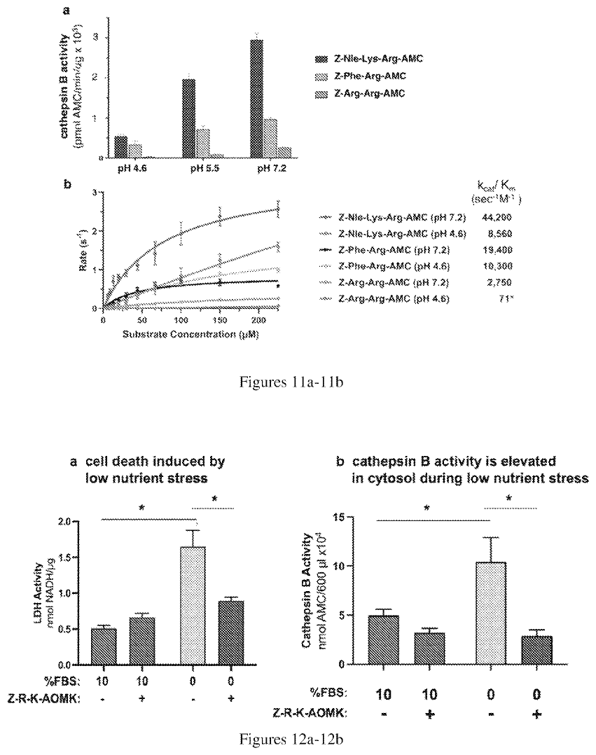

Specific cathepsin B activity in neuronal and glia cells assessed by Z-Nle-Lys-Arg-AMC substrate. To evaluate the novel substrate Z-Nle-Lys-Arg-AMC in a complex biological sample that contains other proteases and proteins, assay of cathepsin B activity in homogenates of human neuroblastoma cells (SHSY-5Y and SK-N-MC neuroblastoma) and mouse microglia cells (BV2 microglia) compared use of the Z-Nle-Lys-Arg-AMC substrate to Z-Arg-Arg-AMC and Z-Phe-Arg-AMC substrates. Cathepsin B activity was assessed as CA-074 sensitive activity (FIGS. 6a-6c), ensuring that the observed activity was inhibited by the specific CA-074 inhibitor of cathepsin B (52, 53). For the three cell types, the highest specific activity of cathepsin B was observed at pH 5.5 compared to pH 4.6 and pH 7.2 (FIGS. 10a-10c). Cathepsin B is present in situ within the pH 5.5 environment of secretory vesicles that produce peptide neurotransmitters (54).

The two neuroblastoma cell lines showed similar specific activities of cathepsin B. The microglia cells displayed high cathepsin B specific activity that was 2 to 4 times higher than the neuroblastoma cells depending on the pH. Notably, the Z-Arg-Arg-AMC substrate detected none or little activity at pH 4.6 in the three cell types, whereas the Z-Nle-Lys-Arg-AMC substrate detected robust cathepsin B activity.

To assess the prediction that mouse cathepsin B resembles human cathepsin B for substrate preferences as demonstrated by the cellular assays, purified recombinant mouse cathepsin B was evaluated with the Z-Nle-Lys-Arg-AMC, Z-Arg-Arg-AMC substrates, as well as the Z-Phe-Arg-AMC substrates at pH 4.6, 5.5, and 7.2 (FIGS. 11a-11b). The highest specific activity of mouse cathepsin B was observed with Z-Nle-Lys-Arg-AMC among the three substrates. Z-Arg-Arg-AMC cleaving activity of cathepsin B was low. A moderate specific activity was observed with Z-Phe-Arg-AMC. These activities of mouse cathepsin B with the three substrates were represented by kcat/Km kinetic values showing high catalytic efficiency for the Z-Nle-Lys-Arg-AMC substrate, modest catalytic efficiency for Z-Phe-Arg-AMC, and low catalytic efficiency for ZArg-Arg-AMC substrate (FIGS. 11a-11b). These findings demonstrate Z-Nle-Lys-Arg-AMC as an excellent substrate for mouse cathepsin B, as well as human cathepsin B.

Higher abundance of cathepsin B protein in microglia compared to neuroblastoma cells represented by cathepsin B activities. To assess whether the higher cathepsin B specific activity in microglia compared to neuroblastoma cells may related to greater levels of cathepsin B enzyme protein, the relative levels of cathepsin B protein in these cells were evaluated by proteomics mass spectrometry (Table 2). Label-free proteomics identified and quantitated the relative levels of the cysteine cathepsins. The level of cathepsin B in microglia cells was about 3-fold greater than in the two neuroblastoma cell types, thus, correlating with the observation of an average of about 3 times greater cathepsin B activity in microglia compared to neuroblastoma cells.

TABLE 2
Examples of variant inhibitors with N- and C-groups
of Nle-Lys-Arg for inhibition of cathepsin B.

| N-Cap group | C-warhead group, active pharmaceutical ingredient |
|---|---|
| acetyl | aldehyde |
| alkyne | acyloxymehyl ketone (AOMK) |
| carboxybenzyl (Z) | chloromethyl ketones |
| morpholino | epoxide |
| | halomethyl ketone |
| | maleimides |
| | sulfonate esters |
| | vinyl sulfone |

It was of interest to observe that the microglia cells contained higher levels of multiple cysteine cathepsins compared to the neuroblastoma cells. The microglia cells contained robust levels of cathepsins B, D. and X, combined with cathepsins A, K, L, and C. In the SH-SY5Y neuroblastoma cells, only two cathepsins, B and D, were detected; in the SKNMC neuroblastoma cells cathepsins B and D were abundant and low levels of cathepsin C was detected.

These findings that differing levels among cathepsin B and related cysteine cathepsins exist in different cell types. Importantly, the new Z-Nle-Lys-Arg-AMC substrate can detect related levels of cathepsin B activity. These results show that Z-Nle-Lys-Arg-AMC robustly monitors cathepsin B specific activity representing enzyme protein levels.

The prominent roles of cathepsin B in health and disease occur in acidic lysosomes and at the neutral pH of cytosol, nuclei, and extracellular locations. Notably, cathepsin B displays different substrate cleavage properties at the acidic compared to neutral pH conditions. It is, therefore, necessary to develop specific substrates for cathepsin B that specifically measure its proteolytic activity over broad pH ranges. Evaluation by this study of current substrates used to monitor cathepsin B activity, consisting of Z-Phe-Arg-AMC and Z-Arg-Arg-AMC, found that they lack specificity since they are cleaved by other cysteine cathepsins; furthermore, Z-Arg-Arg-AMC does not optimally assess cathepsin B activity at acidic pH. Therefore the purpose of study was to conduct in-depth substrate cleavage profiles of cathepsin B compared to other cysteine cathepsins K, L, S, V, and X for design of specific fluorogenic substrates that can monitor cathepsin B proteolytic activity at acidic to neutral pH conditions. Cleavage profiles of these proteases were assessed by MSP-MS that analyzed cleavages of peptide library components at pH 4.6 and pH 7.2. Analysis of the preferred and non-preferred residues at the P3, P2, and P1 positions adjacent to the P1-P1′ cleavage sites predicted the tripeptide Z-Nle-Lys-Arg-AMC as a preferred substrate for cathepsin B. Significantly, Z-Nle-Lys-Arg-AMC displayed the advantageous properties of measuring high cathepsin B specific activity over acidic to neutral pHs and was specific for cathepsin B over other cysteine cathepsins. Z-Nle-Lys-Arg-AMC specifically measured cathepsin B activity in neuronal and glia cells which were consistent with relative abundances of cathepsin B protein. These findings validate Z-Nle-Lys-Arg-AMC as a novel substrate that specifically monitors cathepsin B activity over a broad pH range.

Substrate cleavage profiling analysis of cathepsin B and cysteine cathepsins conducted by MSP-MS, at pH 4.6 and pH 7.2, revealed each protease's distinct pattern of preferred amino acids at P4 to P4′ positions of the P1-P1′ cleavage sites of the peptide library. We focused on the P3 to P1 residue preferences for design of Z-peptide-AMC substrates that utilize non-prime site residues of cleavage sites. Comparisons showed that cathepsin B displayed unique preferences over the other cysteine cathepsins, consisting of Nle at the P3 position, Lys at the P2 position, and Arg at the P3 position at both acidic and neutral pHs. This evaluation formed the basis for design of Z-Nle-Lys-Arg-AMC as a specific cathepsin B substrate. Indeed, assessment of Z-Nle-Lys-Arg-AMC with a series of Z-peptide-AMC substrates indicated that Nle was important over other substitutions consisting of Leu, Tyr, Phe, Ala, Trp, Gly, or Val. Dipeptides containing dibasic sequence combinations of Lys and Arg were poor compared to Z-Nle-Lys-Arg-AMC as substrate. The pH profile of Z-Nle-Lys-Arg-AMC detected cathepsin B of high specific activity at pH 4.5 to pH 7.5, which covers the biological pH range of cathepsin B within acidic lysosomes to modestly acidic secretory vesicles and neutral cytosol, nuclei, and extracellular locations.

The high catalytic activity and specificity of Z-Nle-Lys-Arg-AMC was based on the preference of cathepsin B for P3 and P2 residues adjacent to the P1-P1′ cleavage site. When compared to the Z-dipeptide-AMC substrates, the Z-tripeptide-AMC variants with inclusion of Nle as the P3 residue enhances cathepsin B specificity and catalytic efficiency as shown by the higher specific activities for Z-Nle-Lys-Arg-AMC and Z-Nle-Arg-Arg-AMC compared to their dipeptide variants ZLys-Arg-AMC and Z-Arg-Arg-AMC, respectively. Notably. Val or Gly at P3 prevented any turnover of the substrate, suggesting that the P3 residue interacting with the enzyme S3 pocket plays a key role in substrate binding even when favorable amino acids are present at other positions. Additionally, at the P2 position, cathepsin B preferred Lys and Arg basic residues a both acidic pH 4.6 and neutral pH 7.2.

In neuronal and glia cells, cathepsin B activity was measured with the high specific activity with the novel substrate Z-Nle-Lys-Arg-AMC. Cathepsin B activity was confirmed by inhibition by CA-074, a specific inhibitor of cathepsin B (52, 53), indicating CA-074-sensitive activity. While Z-Nle-Lys-Arg-AMC was highly sensitive for monitoring cathepsin B activity, the Z-Arg-Arg-AMC could monitor only low cathepsin B activity. The Z-Phe-Arg-AMC substrate detected cathepsin B as well as other cysteine cathepsin activity and, thus, was not specific for cathepsin B.

These findings show that the novel Z-Nle-Lys-Arg-AMC substrate is specific for cathepsin B over other cysteine cathepsins and is a sensitive substrate to monitor cathepsin B activity over a broad pH range from acidic to neutral conditions. This substrate is advantageous over other routinely used substrates for cathepsin B which are non-specific or unable to detect the enzyme's activity at low to high pH conditions. Z-Nle-Lys-Arg-AMC detects cathepsin B in human and mouse species cell types and thus will be useful for clinical cathepsin B biomarker studies as well as for mouse models of human disease conditions.

### Materials and Methods

Materials: Cathepsin B and cysteine cathepsins, substrates, MSP-MS library and nano-LCMS/MS, cell culture, and proteomics. Cathepsin B and cysteine cathepsins (human, recombinant), were obtained from R & D Systems (Minneapolis. MN) or Abcam (Cambridge, MA). Proteases obtained for this study were procathepsin B (R & D Systems, #953-CY-010), active cathepsin L (R & D Systems, #952-CY-010), active cathepsin K (Abcam, #ab157067), procathepsin S (R & D Systems, #1183-CY-010), procathepsin V (R & D Systems, #1080-CY-010), and procathepsin X (R & D, #934-CY). Mouse cathepsin B was obtained from R & D Systems (R & D Systems, #965-CY-010). 5 The UniProt identification numbers for the human cathepsin proteases studied are P07858 for cathepsin B, P07711 for cathepsin L, P43235 for cathepsin K, P25774 for cathepsin L, 060911 for cathepsin V, and Q9UBR2 for cathepsin X (also known as cathepsin Z). The UniPro identification for mouse cathepsin B is P10605.

The substrates Z-KR-AMC, Z-GKR-AMC, Z-VKR-AMC, Z-YKR-AMC, Z-FKR-AMC, Z-WKRAMC, Z-LKR-AMC, Z-AKR-AMC, Z-nRR-AMC, and Z-nKR-AMC (“n” represents norleucine) were custom synthesized by GenScript (Piscataway, NJ). Z-RR-AMC was purchased from Bachem (#4004789) (Torrance. CA). Z-FR-AMC was purchased from Anaspec (#AS-24096) (Fremont, CA).

The design and synthesis of the 228 14-mer peptide library used for MSP-MS assays have been described (51). MSP-MS assays utilized low-bind 600 μL microtubes (Corning, Reynosa, MX), dithiothreitol (DTT) (#V351, Promega, Madison, WI), urea (#U2222, Teknova, Hollister, CA), HPLC-grade water (#W6-4, Fisher Scientific), citric acid monohydrate (#1.00244.0500, Merck, Burlington, MA), sodium phosphate dibasic anhydrous (#SX-072305, EMD, Burlington, MA), sodium acetate (#BP-333-500, Fisher Scientific, Fair Lawn, NJ), EDTA (#324503, Calbiochem, Burlington, MA), sodium chloride (#S271-1, Fisher Chemical, Pittsburgh, PA), acetonitrile (#A955-4, Fisher Chemical, Pittsburgh, PA), formic acid (FA) (#A117-50, Fisher Chemical, Pittsburgh, PA), trifluoroacetic acid (TFA) (#A116-50, Fisher Chemical, Pittsburgh, PA), C18 LTS Tips (#PTLC18-960, Rainin. Oakland. CA), C18 for SPE stage-tips (#2215-C18, 3M Co., Maplewood, MN), and BEH C18 packing material (#186004661, Waters Corp., Milford, MA).

Cell culture of human neuroblastoma (SHSY-5Y and SK-N-MC) and mouse microglia (BV2) utilized reagents consisting of RPMI 1640, MEMa, F-12K, heat-inactivated fetal bovine serum, and phosphate-buffered saline (PBS) (catalogue numbers 11875093, 12561056, 30-2004, and 10082147, 10010023, respectively, from Gibco, Grand Island, NY). For proteomics, cells were homogenized in buffer containing a cocktail of protease inhibitors of pepstatin A, leupeptin, chymostatin, and AEBSF (catalogue numbers 516481, 108976, 230790, 101500, respectively from Millipore Burlington, MA), and E64c (catalogue number N-1655 from Bachem, Torrance, CA).

Proteomics of cells utilized Trypsin/Lys-C (V5037, Promega Madison WI), empore C18 (octadecyl) (2215, from 3M, St, Paul, MN), peptide assay (23275, Pierce Waltham, MA) for sample preparation, combined with nano-LC-MS/MS reagents of trifluoroacetic acid, formic acid, Optima LCMS water, Optima LCMS acetonitrile (A116, 85178, W65, A955, respectively, from Fisher Scientific, Waltham, MA). LC columns utilized were pricofrit self-pack column of 360 μm OD, 75 μm ID, 15 μm tip (PF360-75-15-N from New Objective, Littleton, MA) and Acquity UPLC BEH C18 (130 Å, 1.7 μm) (186004661, from Waters Copr. Meilford, MA).

Activation of cathepsin B and cysteine cathepsin proteases. Recombinant human procathepsins B, V, and S were activated by incubation at 37° C. for 30 min in 20 mM Na-acetate pH 5.5, 100 mM NaCl, 5 mM DTT, 1 mM EDTA activation buffer. Recombinant human procathepsin X was activated by incubation at room temperature for 5 min in 20 mM citrate phosphate pH 3.5, 100 mM NaCl and 5 mM DTT activation buffer.

Cathepsin B activity with Z-Arg-Arg-AMC and Z-Phe-Arg-AMC substrates at pH 4.6 and 7.2. Cathepsin B (0.04 ng/μL) activity was assessed with Z-Arg-Arg-AMC and Z-Phe-Arg-AMC substrates (40 μM final concentration) (Table 1) in 40 mM citrate phosphate buffer pH 4.6 or Tris-HCl pH 7.2 in 1 mM EDTA, 100 mM NaCl, and 5 mM DTT with incubation at room temperature (25° C.) in triplicate and AMC fluorescence readings per second (RFU/s) (excitation 360 nm, emission 460 nm) were recorded over a period of 30 min by BioTek Synergy HTX plate reader (Software Version 3.08.01). Enzyme initial velocity was calculated using the highest slope recorded for 10 consecutive fluorescent readings within the initial 30 min. The mean and standard deviation (SD) were determined from triplicates. RFU/s were converted to pmol/min/μg (specific activity) using AMC standard curves. All data were plotted, calculated, and analyzed using GraphPad Prism (Version 9.4.1).

Protease substrate cleavage profiling by multiplex substrate profiling by mass spectrometry (MSP-MS). Peptide library for substrate profiling by mass spectrometry. MSP-MS was performed for cathepsins B, L, K, S, and V at pH 4.6 and pH 7.2 by methods that we have described previously (51). Cathepsin X was performed only at pH 4.6. In a total volume of 22 μL, cathepsin B (0.1 ng/L), cathepsin L (0.04 ng/μL), cathepsin K (0.07 ng/μL), cathepsin S (1.2 ng/μL), cathepsin V (0.16 ng/μL) and cathepsin X (0.09 ng/μL) were incubated with a mixture of 228 14-mer peptides (0.5 μM for each peptide) in 50 mM citrate phosphate at pH 4.6 or pH 7.2, 100 mM NaCl, 5 mM DTT, 1 mM EDTA assay buffer for 15 and 60 min at 25° C. 10 μL was removed at 15 and 60 min to be combined with 60 μL of 8 M urea. A control assay used inactivated cathepsins by incubating it with 8 M urea for 60 min at 25° C. prior to addition of the peptide library in assay buffer. Assays were conducted in quadruplicates. Samples were acidified by addition of 40 μL of 2% TFA and desalted using custom-made C18 spin tips. The collected liquid samples were dried completely in a vacuum centrifuge, and then stored at −70° C. For LC-MS/MS analysis, dried samples were resuspended in 40 μL of 0.1% TFA and 4 μL was injected into the Thermo Fisher Scientific QExactive mass spectrometer with an Ultimate 3000 HPLC. Peptides were separated using reverse phase chromatography by C18 column at 65° C.) conducted as we have previously described (50, 51).

PEAKS bioinformatics analysis. PEAKS (v 8.5) software (Bioinformatics Solutions Inc.) was used for MS/MS data analysis. The 228 14-mer library sequence was used as the database for MS data searching and matching, and as a control, it was also searched with a decoy consisting of the 228 14-mer library sequences in reversed order. A precursor tolerance of 0.01 Da and 20 ppm for MS2 fragments was used. Data were filtered to 0.9% peptide sequence false discovery rates determined from hits from decoys. Label-free quantification was used for peptide quantification and the data was normalized by LOESS-G normalization method using Normalyzer tool version 1.1.1 (55). Outliers from groups of replicates were removed by Dixon's Q test (56). Missing and zero values were treated the same and replaced with imputed values determined from randomized normally distributed numbers within the range of the smallest 5% dataset±SD. The control 0 min values in MSP-MS obtained for cathepsins B, L, K, S, V, and X at pH 4.6 were also analyzed by PEAKS (for n=20). To determine if the peptide intensity data in at least one of the groups of the 0, 15, and 60 min timepoints were statistically significant, ANOVA testing with multiple testing correction was used; those with q<0.05 were considered for further analysis. Cleaved peptide products at the 15 min or 60 min timepoints were defined as having intensity scores of 8-fold or more with p<0.05 by the two-tailed homoscedastic t-test.

Cleavage site analysis by iceLogo. Evaluation of the frequencies of the P4 to P4′ amino acids adjacent to the cleavage sites was conducted using the iceLogo software 1.3.8 where the “experimental data set” consists of the detected cleavage sites defined earlier and the “reference data set” that consists of all possible cleavages within the MSP-MS library of 228 14-mer peptides. z-scores were calculated by the equation (X−μ)/σ, where X is the frequency of the amino acid occurring in the “experimental data set” at a specific position relative to the cleavage site (e.g. P3, P2, P1), μ is the frequency of the amino acid at a specific position in the “reference data set”, and σ is the SD. z-scores were utilized to generate iceLogo illustrations of the relative frequencies of amino acid residues at each of the P3 to P1 positions of the cleaved peptides where heights of the single letter amino acids represent “percent difference”, defined as the difference in frequency for an amino acid appearing in the “experimental data set” relative to the “reference data set”. Amino acids shown above the midline have positive z-scores, indicating preferred amino acids and amino acids shown below the midline have negative z-scores, indicating not preferred amino acids, illustrated using p<0.30 cutoff criteria in the iceLogo software with p<0.05 labeled in purple.

Analysis of pH-dependent cathepsin B activity with fluorogenic substrates designed from MSPMS data. For comparison of cathepsin B activity at pH 4.6, pH 5.5, and pH 7.2 with multiple fluorogenic substrates, cathepsin B (0.04 ng/μL) was incubated with 40 μM substrates consisting of Z-Lys-Arg-AMC. Z-Phe-Arg-AMC. Z-Arg-Arg-AMC. Z-Gly-Lys-Arg-AMC, Z-Val-Lys-Arg-AMC, Z-Tyr-Lys-Arg-AMC. Z-Phe-Lys-Arg-AMC, Z-Trp-Lys-Arg-AMC, Z-Leu-Lys-Arg-AMC, Z-Ala-Lys-Arg-AMC, Z-Nle-Arg-Arg-AMC, and Z-Nle-Lys-Arg-AMC. AMC product formation (RFU/s) was monitored in 40 mM citrate phosphate, pH 4.6 and pH 7.2, 1 mM EDTA, 100 mM NaCl, and 5 mM DTT was determined from the average of RFU/s triplicates at each pH condition.

For analysis of pH-dependent activity cathepsin B (0.04 ng/μL), proteolytic activity was monitored over the pH range of pH 2.2-9.0 in 40 mM citrate phosphate (pH 2.2 to pH 6.6) and 40 mM Tris-HCl (pH 7.0 to 9.0), 1 mM EDTA, 100 mM NaCl, and 5 mM DTT, with preincubation in each pH buffer for 10 min prior to initiating the assay by addition of Z-Nle-Lys-Arg-AMC, Z-Arg-Arg-AMC, or Z-Phe-Arg-AMC to a final concentration of 40 μM to generate the pH curve data.

For evaluation of cathepsin B and cysteine cathepsins for substrate selectivity each cathepsin was incubated with 4 μM substrates consisting of Z-Phe-Arg-AMC. Z-Arg-Arg-AMC, and Z-Nle-Lys-Arg-AMC, activity was monitored in 40 mM citrate phosphate (pH 4.6, pH 5.5 and pH 7.2), 1 mM EDTA, 100 mM NaCl, and 5 mM DTT for cathepsin B (0.04 ng/μL), cathepsin K (0.03 ng/μL), cathepsin L (0.03 ng/μL), cathepsin S (0.14 ng/μL), cathepsin V (0.04 ng/μL), and cathepsin X (0.20 ng ng/μL). Since cathepsin X cannot cleave any of the AMC substrates, MCARPPGFSAFK(Dnp)-OH (R & D Systems #ES005) was used to verify that cathepsin X was active.

Kinetic parameters of cathepsin B assessed by kcat and Km values with fluorogenic substrates. The kinetic parameters of kcat and Km for Z-Nle-Lys-Arg-AMC, Z-Arg-Arg-AMC, and Z-Phe-Arg-AMC substrates were determined at pH 4.6 and pH 7.2, using substrate concentrations of 225-5.9 μM with 0.04 ng/L cathepsin B. RFU values were converted to s−1 using AMC standard curves. The kcat and Km values were obtained from curve fitting the converted RFU data using GraphPad Prism9 software to the equation v0=(Vmax[S])/(Km+[S]) where v0 is the initial velocity of the enzyme with its corresponding [S] substrate concentration and Vmax is the maximum enzyme velocity at saturated [S]. Vmax=kcat[E]T, where [E]T is the total cathepsin B concentration used in the assay. Km is the x-axis value [S] where y=Vmax/2. SD values for kcat and Km were determined from curving fitting the v0 and [S] data from triplicates.

Neuronal and microglia cell culture. Human neuroblastoma SH-SY5Y (57) and SK-N-MC (58) and embryonic mouse microglia BV2 (gifted by Christopher Glass, Ph.D, UC San Diego) were grovwn in 5% CO2 and at 37° C. under sterile conditions. Growth media conditions were 10% heat inactivated fetal bovine serum (Life Technologies, Carlsbad, CA), and media consisting of 90% MEMa (Life Technologies) for SK-N-MC cells, 45% Ham's F12-K (ATCC, Manassas, VA) and 45% MEMa (Life Technologies) for SH-SY5Y, and 90% RPMI (Life Technologies) BV2.

Cathepsin B activity in human neuroblastoma and mouse microglia. Characterization of Z-Nle-Lys-Arg-AMC substrate in proteolytic activity assays for specific cathepsin B activity of cell homogenates (prepared in 0.32 M sucrose) was conducted at 40 mM citrate phosphate pH 4.6, 5.5, and 7.2, 5 mM DTT, 1 mM EDTA, 100 mM NaCl, 1.2% DMSO, and homogenates of human neuroblastoma cell lines, SHSY-5Y and SK-N-MC, and mouse microglia cell line, BV2. A concentration of 6 μM Z-Nle-Lys-Arg-AMC, Z-Arg-Arg-AMC, and Z-Phe-Arg-AMC substrates were used in the proteolytic activity assays. Cell homogenates were diluted 1:10 prior to addition to assays. Homogenate protein concentrations were measured by the Biorad protein assay kit (Biorad #5000113, Hercules, CA) The cathepsin B specific inhibitor, CA-074, was used to specifically inhibit cathepsin B activity at concentrations 1 μM at pH 4.6 and 5.5, and 1 μM at pH 7.2. Assays were performed in 96-well plates at room temperature (25° C.) in a total volume of 100 μl with triplicates. Cell homogenates were incubated with CA-074 or inhibitor controls at RT for 30 minutes in the absence of substrate, followed by addition of substrate and incubation at 37° C. Fluorescence was quantified at 15, 30, and 60 minutes after substrate was added, by a Biotek Synergy HTX microplate reader with excitation at 360 nm, and emission at 460 nm, gain 50, top optics and read height at 1 mm. Prism GraphPad software was used to analyze data.

Proteomics identification and quantitation of cysteine cathepsins in neuronal and microglia cells. Analysis of neuronal and microglia cells by proteomics mass spectrometry utilized protocols that we have previously described (59, 60). To summarize, approximately 108 cells were collected from 90% confluent flasks with PBS washing and centrifugation at 500×g. Cell pellets were lysed by dounce homogenization in ice-cold 100 mM Tris pH 7.4, 50 mM NaCl, 1 mM EDTA and protease inhibitors (10 mM Pepstatin A, 10 mM Leupeptin, 10 mM Chymostatin, 100 mM AEBSF (Millipore, Burlington, MA) and 10 mM E64c (Bachem, Torrance, KA)). Tryptic digests for proteomics analyses were prepared from 200 mg of total cellular protein as described previously (59), with two biological flask replicates per cell line, and triplicate technical replicate injections per biological replicate. Tryptic peptides were diluted to 0.5 mg/ml in 2% acetonitrile, 0.1% trifluoroacetic acid, and 2 mg total amount injected by nano-LC-MS/MS on an Dionex UltiMate 3000 nano LC and Q-Exactive mass spectrometer (Thermo Fisher Scientific). Spectra were acquired as described previously (59). For protein identification and quantitation, spectra were queried against a custom database consisting of the protein sequences of 15 human cathepsins (SH-SY5Y and SK-N-MC) or 15 mouse cathepsins (BV2) with PEAKS v. 8.5 using decoy-fusion and label-free quantification (LFQ) methods (Bioinformatics Solutions Inc., Waterloo, ON), with search parameters described previously (59). Proteins were considered identified if present in at least 2 of 3 technical replicates and at least 1 biological replicate. Proteins were considered quantifiable based on the same criteria as for identification. Peak intensity areas of quantifiable data are expressed as mean+SD.

Data Availability. All mass spectrometry data have been deposited into massive.ucsd.edu under identifier MSV000090043 and proteomeXchange under identifier PXD035641.

Cathepsin B Selective Substrate Z-Nle-Lys-Arg-AMC Demonstrates Elevated Cathepsin B Activity in Cellular Cell Death and in Mouse Brain after Traumatic Brain Injury.

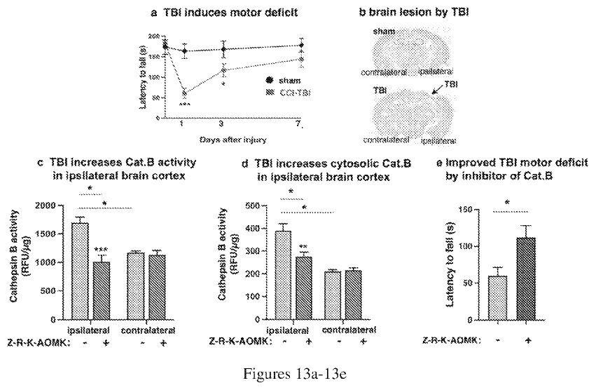

Z-Nle-Lys-Arg-AMC demonstrates elevated cathepsin B activity in cellular cell death. The Z-Nle-Lys-Arg-AMC (Z-nKR-AMC) substrate has been demonstrated as a specific substrate for measuring cathepsin B proteolytic activity (62). Data shows that cytosolic cathepsin B activity, measured by the Z-nKR-AMC substrate, is elevated during cell death induced by low nutrient stress (0% fetal bovine serum, FBS, compared to normal 10% FBS) in microglia cells (in culture) (FIGS. 12a and 12b). Furthermore, inhibition of elevated cathepsin B by Z-Arg-Lys-AOMK resulted in decreased cell death (FIGS. 12a and 12b). This data shows that the Z-nKR-AMC substrate measures increased cathepsin B during cell death in microglia cells.

Regulation of cathepsin B activity during cell death induced by low nutrient stress in microglia cells. FIG. 12a shows cell death in microglia cells induced by low nutrient stress. Microglia cells (mouse BV2 cells) were treated with low nutrient culture media (0% fetal bovine serum, FBS) compared to a normal nutrient condition (10% FSB) for 24 hours. The low nutrient stress condition induced cell death monitored by LDH (lactate dehydrogenase) released into the culture media. The stress-induced increase in cell death was reduced by the inhibitor Z-Arg-Lys-AOMK (2 μM), a selective inhibitor of cathepsin B. (n=6, mean+sem, *p<0.05, student's t-test). FIG. 12b shows cathepsin B activity is increased in the cytosol of microglia cells during low nutrient stress. Cathepsin B activity was elevated in cytosol isolated from microglia cells after treatment with low nutrient stress (0% FBS) for 24 hours. Cathepsin B activity was measured with the specific substrate Z-Nle-Lys-Arg-AMC (60 mM in assay). Furthermore, the increase in cytosolic cathepsin B activity was blocked by the cathepsin B inhibitor Z-Arg-Lys-AOMK (2 μM) during low nutrient stress. (n=6, mean+sem, *p<0.05, student's t-test).

Z-Nle-Lys-Arg-AMC demonstrates involvement of elevated cathepsin B activity in motor deficit induced by traumatic brain injury (TBI) in mouse brain. In mice subjected to TBI by the controlled cortical impact (CCI) model in mice, motor deficit occurred at 1 and 3 days after CCI-TBI, shown by rotarod assays (FIG. 13a). TBI produced cortical brain lesions, illustrated by cortical brain tissue loss at the site of TBI, on the ipsilateral side of the brain (FIG. 13b, tissue loss shown by arrow). TBI resulted in elevation of cathepsin B activity in the ipsilateral brain cortex homogenate compared to the contralateral brain (FIG. 13c); the Z-Nle-Lys-Arg-AMC substrate was used to measure cathepsin B activity. The cytosol fraction of the homogenate was isolated and cathepsin B activity was found to be elevated in the cytosol from the ipsilateral cortex, but not the contralateral cortex of brain (FIG. 13d). Notably, inhibition of cathepsin B activity in brain homogenate and cytosol (FIGS. 13c, 13d) by Z-Arg-Lys-AOMK resulted in improved motor deficit on day 1 after TBI (FIG. 13e).

These findings show that cathepsin B activity, measured by its specific substrate Z-Nle-Lys-Arg-AMC, is elevated in mouse brain after TBI, and inhibition of cathepsin B improves TBI-induced motor deficit.

FIGS. 13a-13e show traumatic brain injury (TBI) in mice results in elevated brain cathepsin B activity, involved in motor deficit. FIG. 13a shows TBI induced motor deficit in mice subjected to the controlled cortical impact (CCI) model of TBI. Mice were subjected to the controlled cortical impact (CCI) model of TBI. Motor function was assessed by the rotarod assay on days 1, 3, and 7 after TBI. Control sham mice received surgery of the scalp but no TBI. Rotarod data are shown as mean+SD (n=5/group, mean+sem, *p<0.05, student's t-test). FIG. 13b shows brain lesion induced by TBI. The TBI injury was conducted on the ipsilateral side of the brain cortex, shown by the tissue lesion at the site of TBI impact. FIG. 13c shows elevated cathepsin B activity in homogenate of mouse ipsilateral brain cortex after TBI. Cathepsin B activity in homogenate of mouse brain cortex was measured with the Z-Nle-Lys-Arg-AMC substrate on day 6 after TBI. These TBI mice were treated with vehicle or Z-Arg-Lys-AOMK inhibitor (20 mg/kg, ip). (n=6 per group for vehicle-treated, and n=4 per group for treatment with inhibitor Z-Arg-Lys-AOMK; mean+sem, p<0.05, student's t-test).

FIG. 13d shows elevation of cytosolic cathepsin B activity in mouse ipsilateral brain cortex after TBI. Cytosol from brain homogenate (obtained on day 6 after TBI) was isolated by differential density centrifugation. Cathepsin B in the cytosol from brain cortex was measured by the substrate Z-Nle-Lys-Arg-AMC substrate. (n=6 per group for vehicle treated, and n=4 per group for treatment with inhibitor Z-Arg-Lys-AOMK: mean+sem, p<0.05, student's t-test).

FIG. 13e shows inhibitor of cathepsin B improves motor deficit induced by TBI. TBI mice were treated with Z-Arg-Lys-AOMK inhibitor (20 mg/kg, ip) one day before TBI, on the day of TBI, and on day 1 after TBI. Motor function was measured on day 1 after TBI. (n=6 per group for vehicle-treated, and n=4 per group for treatment with inhibitor Z-Arg-Lys-AOMK; mean+sem, p<0.05, student's t-test).

### REFERENCES

1. De Duve C, Wattiaux R. (1966) Functions of lysosomes. Annu Rev Physiol. 28, 435-92.
2. Xu H, Ren D. (2015) Lysosomal physiology. Annu Rev Physiol. 77, 57-80.
3. Lawrence R E, Zoncu R. (2019) The lysosome as a cellular centre for signaling, metabolism and quality control. Nat Cell Biol. 21, 133-142.
4. Turk, V., Stoka, V., Vasiljeva. O., Renko, M., Sun, T., Turk, B., Turk, D. (2012) Cysteine cathepsins: from structure, function and regulation to new frontiers, Biochim Biophys Acta. 1824, 68-88.
5. Barrett, A. J., Rawlings, N. D., and Woessner, J. F. (2004). Handbook of Proteolytic Enzymes, 2nd edition, Elsevier Academic Press, Amsterdam.
6. Mindell, JA. (2012) Lysosomal acidification mechanisms. Annu Rev Physiol. 74, 69-86.
7. Ishida, Y., Nayak, S., Mindell, J. A., Grabe, M. (2013) A model of lysosomal pH regulation, J Gen Physiol. 141, 705-20.
8. Ohkuma, S., Poole, B. (1978) Fluorescence probe measurement of the intralysosomal pH in living cells and the perturbation of pH by various agents, Proc Natl Acad Sci USA 75, 3327-31.
9. Hook G, Reinheckel T, Ni J, Wu Z, Kindy M, Peters C, Hook V. (2022) Cathepsin B Gene Knockout Improves Behavioral Deficits and Reduces Pathology in Models of Neurologic Disorders, Pharmacol Rev. 74, 600-629.
10. Nakanishi, H. (2020) Microglial cathepsin B as a key driver of inflammatory brain diseases and brain aging, Neural Regen Res. 15, 25-29.
11. Ditaranto, K., Tekirian, T. L., Yang, A. J. (2001) Lysosomal membrane damage in soluble Abeta-mediated cell death in Alzheimer's disease, Neurobiol Dis. 8, 19-31.
12. Umeda, T., Tomiyama, T., Sakama, N., Tanaka, S., Lambert, M. P., Klein, W. L., Mori, H. (2011) Intraneuronal amyloid Q oligomers cause cell death via endoplasmic reticulum stress, endosomal/lysosomal leakage, and mitochondrial dysfunction in vivo, J Neurosci Res. 89, 1031-42.
13. Lafrenaye, A. D., McGinn. M. J., Povlishock, J. T. (2012) Increased intracranial pressure after diffuse traumatic brain injury exacerbates neuronal somatic membrane poration but not axonal injury: evidence for primary intracranial pressure-induced neuronal perturbation, J Cereb Blood Flow Metab. 32, 1919-32.
14. Luo, C. L., Chen, X. P., Li, L. L., Li, Q. Q., Li, B. X., Xue, A. M., Xu, H. F., Dai, D. K., Shen, Y. W., Tao, L. Y., Zhao, Z. Q. (2013) Poloxamer 188 attenuates in vitro traumatic brain injury-induced mitochondrial and lysosomal membrane permeabilization damage in cultured primary neurons, J Neurotrauma 30, 597-607.
15. Hernandez, M. L.; Marone, M.; Gorse, K. M.: Lafrenaye, A. D. Cathepsin B Relocalization in Late Membrane Disrupted Neurons Following Diffuse Brain Injury in Rats. ASN Neuro. 2022, 14, 17590914221099112.
16. Windelbom, J. A., Lipton, P. (2008) Lysosomal release of cathepsins causes ischemic damage in the rat hippocampal slice and depends on NMDA-mediated calcium influx, arachidonic acid metabolism, and free radical production, J Neurochem. 106, 56-69.
17. Kilinc, M., Gürsoy-Ozdemir, Y., Gurer, G., Erdener, S. E., Erdemli, E., Can, A., Dalkara, T. (2010) Lysosomal rupture, necroapoptotic interactions and potential crosstalk between cysteine proteases in neurons shortly after focal ischemia. Neurobiol Dis. 40, 293-302.
18. Dong, H., Qin, Y., Huang, Y., Ji, D., Wu, F. (2019) Poloxamer 188 rescues MPTP-induced lysosomal membrane integrity impairment in cellular and mouse models of Parkinson's disease. Neurochem Int. 126, 178-186.
19. Freeman, D., Cedillos, R., Choyke, S., Lukic, Z., McGuire, K., Marvin, S., Burrage, A. M., Sudholt, S., Rana, A., O'Connor. C., Wiethoff, C. M., Campbell, E. M. (2013) Alpha-synuclein induces lysosomal rupture and cathepsin dependent reactive oxygen species following endocytosis, PLoS One 8, e62143.
20. Amritraj, A., Peake, K., Kodam, A., Salio, C., Merighi, A., Vance, J. E., Kar, S. (2009) Increased activity and altered subcellular distribution of lysosomal enzymes determine neuronal vulnerability in Niemann-Pick type Cl-deficient mice, Am J Pathol 175, 2540-56.
21. Fujisawa, A., Kambe, N., Saito, M., Nishikomori, R., Tanizaki, H., Kanazawa, N., Adachi, S., Heike, T., Sagara, J., Suda, T., Nakahata, T., Miyachi, Y. (2007) Disease-associated mutations in CIAS1 induce cathepsin B-dependent rapid cell death of human THP-1 monocytic cells, Blood 109, 2903-11.
22. Rajamaki, K., Lappalainen, J., Oomi, K., Valimaki, E., Matikainen, S., Kovanen, P. T., Eklund, K. K. (2010) Cholesterol crystals activate the NLRP3 inflammasome in human macrophages: a novel link between cholesterol metabolism and inflammation. PLoS One 5, e11765.
23. Gonzalez, E. A., Martins, G. R., Tavares, A. M. V., Viegas, M., Poletto, E., Giugliani, R., Matte. U., Baldo. G. (2018) Cathepsin B inhibition attenuates cardiovascular pathology in mucopolysaccharidosis I mice, Life Sci 196, 102-109.
24. Saluja, A., Dudeja, V., Dawra, R., Sah, R. P. (2019) Early Intra-Acinar Events in Pathogenesis of Pancreatitis, Gastroenterology 156, 1979-1993.
25. Amaral, E. P., Riteau, N., Moayeri, M., Maier, N., Mayer-Barber, K. D., Pereira, R. M., Lage, S. L., Kubler, A., Bishai, W. R., D'Império-Lima, M. R., Sher, A., Andrade, B. B. (2018) Lysosomal Cathepsin Release Is Required for NLRP3-Inflammasome Activation by Mycobacterium tuberculosis in Infected Macrophages, Front Immunol 9, 1427.
26. Morchang, A., Panaampon, J., Suttitheptumrong, A., Yasamut, U., Noisakran, S., Yenchitsomanus, P. T., Limjindapom, T. (2013) Role of cathepsin B in dengue virus-mediated apoptosis, Biochem Biophys Res Commun 438, 20-5.
27. Bright, G. R., Fisher, G. W., Rogowska, J., Taylor, D. L. (1987) Fluorescence ratio imaging microscopy: temporal and spatial measurements of cytoplasmic pH, J Cell Biol. 104, 1019-33.
28. Madshus, T. H. (1988) Regulation of intracellular pH in eukaryotic cells, Biochem J. 250, 1-
29. de Castro, M. A., Bunt. G., Wouters, F. S. (2016) Cathepsin B launches an apoptotic exit effort upon cell death-associated disruption of lysosomes, Cell Death Discov 2, 16012.
30. Droga-Mazovec, G., Bojic, L., Petelin, A., Ivanova, S., Romih, R., Repnik, U., Salvesen, G. S., Stoka, V., Turk, V., Turk, B. (2008) Cysteine cathepsins trigger caspase-dependent cell death through cleavage of bid and antiapoptotic Bcl-2 homologues, J Biol Chem 283, 19140-50.
31. Kavcic, N., Pegan, K., Turk, B. (2017) Lysosomes in programmed cell death pathways: from initiators to amplifiers. Biol Chem 398, 289-301.
32. Wei, M. C., Lindsten, T., Mootha, V. K., Weiler, S., Gross, A., Ashiya, M., Thompson, C. B., Korsmeyer, S. J. (2000) tBID, a membrane-targeted death ligand, oligomerizes BAK to release cytochrome c, Genes Dev 14, 2060-71.
33. Campden. R. I., Zhang, Y. (2019) The role of lysosomal cvsteine cathepsins in NLRP3 inflammasome activation, Arch Biochem Biophys 670, 32-42.
34. Bai, H., Yang, B., Yu, W., Xiao, Y., Yu, D., Zhang, Q. (2018) Cathepsin B links oxidative stress to the activation of NLRP3 inflammasome, Exp Cell Res 362, 180-187.
35. Lian, D., Lai, J., Wu, Y., Wang, L., Chen, Y., Zhang, Y., Boini, K. M., Huang, Y., Chen, Y. (2018) Cathepsin B-mediated NLRP3 inflammasome formation and activation in angiotensin II—Induced hypertensive mice: role of macrophage digestion dysfunction, Cell Phvsiol Biochem 50, 1585-1600.
36. Amaral, E. P., Riteau, N., Moayeri, M., Maier, N., Mayer-Barber, K. D., Pereira, R. M., Lage, S. L., Kubler, A., Bishai, W. R., D'Imperio-Lima, M. R., Sher, A., Andrade, B. B. (2018) Lysosomal cathepsin release is required for NLRP3-inflammasome activation by Mycobacterium tuberculosis in infected macrophages. Front Immunol 9, 1427.
37. Jordans, S., Jenko-Kokalj, S., Kahl, N. M., Tedelind, S., Sendt, W., Bromme, D., Turk, D., Brix, K. (2009) Monitoring compartment-specific substrate cleavage by cathepsins B, K, L, and S at physiological pH and redox conditions, BMC Biochem 20, 23.
38. Tedelind, S., Poliakova, K., Valeta, A., Hunegnaw, R., Yemanaberhan, E. L., Heldin, N. E., Kurebayashi, J., Weber, E., Kopitar-Jerala, N., Turk, B., Bogyo, M., Brix, K. (2010) Nuclear cysteine cathepsin variants in thyroid carcinoma cells, Biol Chem 391, 923-35.
39. Hämälistö, S., Stahl, J. L., Favaro, E., Yang, Q., Liu, B., Christoffersen, L., Loos, B., Boldú, C., Joyce, J. A., Reinheckel, T., Barisic, M., Jäättelä, M. (2020) Spatially and temporally defined lysosomal leakage facilitates mitotic chromosome segregation, Nat Commun 11, 229.
40. Cataldo A M, Thayer C Y, Bird E D, Wheelock T R, Nixon R A. (1990) Lysosomal proteinase antigens are prominently localized within senile plaques of Alzheimer's disease: evidence for a neuronal origin. Brain Res. 513, 181-92.
41. Cataldo A M, Hamilton D J, Nixon R A. (1994) Lysosomal abnormalities in degenerating neurons link neuronal compromise to senile plaque development in Alzheimer disease. Brain Res. 640, 68-80.
42. Amritraj A, Hawkes C, Phinney A L, Mount H T, Scott C D, Westaway D, Kar S. (2009) Altered levels and distribution of IGF-II/M6P receptor and lysosomal enzymes in mutant APP and APP+PS1 transgenic mouse brains. Neurobiol Aging. 30, 54-70.
43. Buck, M. R., Karustis, D. G., Day, N. A., Honn, K. V., Sloane, B. F. (1992) Degradation of extracellular-matrix proteins by human cathepsin B from normal and tumour tissues, Biochem J 282, 273-8.
44. Cavallo-Medved, D., Dosescu, J., Linebaugh, B. E., Sameni, M., Rudy, D., Sloane, B. F. (2003) Mutant K-ras regulates cathepsin B localization on the surface of human colorectal carcinoma cells, Neoplasia 5, 507-19.
45. Victor, B. C., Anbalagan, A., Mohamed, M. M., Sloane, B. F., Cavallo-Medved, D. (2011) Inhibition of cathepsin B activity attenuates extracellular matrix degradation and inflammatory breast cancer invasion. Breast Cancer Res 13, R115.
46. Aggarwal, N., Sloane, B. F. (2014) Cathepsin B: multiple roles in cancer, Proteomics Clin Appl 8, 427-37.
47. Bian, B., Mongrain, S., Cagnol, S., Langlois, M. J., Boulanger, J., Bernatchez, G., Carrier, J. C., Boudreau, F., Rivard, N. (2016) Cathepsin B promotes colorectal tumorigenesis, cell invasion, and metastasis, Mol Carcinog 55, 671-87.
48. Caglic D, Kosec G, Bojic L, Reinheckel T. Turk V, Turk B. (2009) Murine and human cathepsin B exhibit similar properties: possible implications for drug discovery. Biol Chem. 390, 175-9.
49. Poreba M, Groborz K, Vizovisek M. Maruggi M, Turk D, Turk B, Powis G. Drag M, Salvesen G S. (2019) Fluorescent probes towards selective cathepsin B detection and visualization in cancer cells and patient samples. Chem Sci. 10, 8461-8477.
50. Yoon M C, Solania A, Jiang Z, Christy M P, Podvin S, Mosier C, Lietz C B. Ito G, Gerwick W H, Wolan D W, Hook G. O'Donoghue A J, Hook V. (2021) Selective Neutral pH Inhibitor of Cathepsin B Designed Based on Cleavage Preferences at Cytosolic and Lysosomal pH Conditions. ACS Chem Biol. 16, 1628-1643.
51. Yoon M C, Christy M P, Phan V V, Gerwick W H, Hook G, O'Donoghue A J, Hook V. (2022) Molecular Features of CA-074 pH-Dependent Inhibition of Cathepsin B. Biochemistry 61, 228-238.
51. O'Donoghue A J, Eroy-Reveles A A, Knudsen G M, Ingram J. Zhou M. Statnekov J B. Greninger A L, Hostetter D R, Qu G, Maltby D A, Anderson M O, Derisi J L, McKerrow J H, Burlingame A L, Craik C S. (2012) Global identification of peptidase specificity by multiplex substrate profiling. Nat Methods. 9, 1095-100.
52. Towatari, T., Nikawa, T., Murata, M., Yokoo, C., Tamai, M., Hanada, K., and Katunuma, N. (1991) Novel epoxysuccinyl peptides. A selective inhibitor of cathepsin B, in vivo, FEBS. Lett. 280, 311-5.
53. Murata, M., Miyashita, S., Yokoo, C., Tamai, M., Hanada, K., Hatayama, K., Towatari, T., Nikawa, T., and Katunuma, N. (1991) Novel epoxysuccinyl peptides. Selective inhibitors of cathepsin B, in vitro, FEBS. Lett. 280, 307-10.
54. Jiang Z, Lietz C B, Podvin S. Yoon M C, Toneff T, Hook V, O'Donoghue A J. (2021) Differential Neuropeptidomes of Dense Core Secretory Vesicles (DCSV) Produced at Intravesicular and Extracellular pH Conditions by Proteolytic Processing. ACS Chem Neurosci 12, 2385-2398.
55. Chawade A, Alexandersson E, Levander F. (2014) Normalyzer: a tool for rapid evaluation of normalization methods for omics data sets. J Proteome Res. 13, 3114-20.
56. Rorabacher, DB. (1991) Statistical treatment for rejection of deviant values: critical values of Dixon's Q parameter and related subrange ratios at the 95% confidence level. Anal. Chem. 63, 139-146.
57. Biedler J L, Roffler-Tarlov S. Schachner M, Freedman L S. (1978) Multiple neurotransmitter synthesis by human neuroblastoma cell lines and clones. Cancer Res. 38, 3751-7. PMID: 29704.
58. Helson L, Das S K, Hajdu S I. (1975) Human neuroblastoma in nude mice. Cancer Res. 1975 September; 35(9):2594-9. PMID: 167965.
59. Podvin S, Jones A, Liu Q, Aulston B, Ransom L, Ames J, Shen G, Lietz C B, Jiang Z, O'Donoghue A J, Winston C, Ikezu T, Rissman R A, Yuan S, Hook V. (2020) Dysregulation of Exosome Cargo by Mutant Tau Expressed in Human-induced Pluripotent Stem Cell (iPSC) Neurons Revealed by Proteomics Analyses. Mol Cell Proteomics 19, 1017-1034.
60. Podvin S, Jones A, Liu Q, Aulston B, Mosier C. Ames J, Winston C. Lietz C B, Jiang Z, O'Donoghue A J, Ikezu T, Rissman R A, Yuan S H, Hook V. (2021) Mutant Presenilin 1 Dysregulates Exosomal Proteome Cargo Produced by Human-Induced Pluripotent Stem Cell Neurons. ACS Omega 6, 13033-13056.
61. Yoon, et al., Distinct Cleavage Properties of Cathepsin B Compared to Cysteine Cathepsins Enable the Design and Validation of a Specific Substrate for Cathepsin B over a Broad pH Range. Biochemistry 2023, 62, 2289-2300 (Jul. 17, 2023).
62. Yoon M C, Phan V, Podvin S, Mosier C, O'Donoghue A J, Hook V. Distinct Cleavage Properties of Cathepsin B Compared to Cysteine Cathepsins Enable the Design and Validation of a Specific Substrate for Cathepsin B over a Broad pH Range. Biochemistry. 2023 Aug. 1:62(15):2289-2300. PMCID: PMC10399199.

1. A peptidic inhibitor of cathepsin B comprising an amino-terminally capped tri-peptide Nle-Lys-Arg.

2. The peptidic inhibitor of cathepsin B of claim 1, wherein the inhibitor is carboxy-terminally capped with AMC.

3. The peptidic inhibitor of cathepsin B of claim 1, wherein the inhibitor is carboxy-terminally capped with an active pharmaceutical ingredient.

4. The peptidic inhibitor of cathepsin B of claim 1, wherein the amino-terminal cap is a benzyloxycarbonyl (Z).

5. The peptidic inhibitor of cathepsin B of claim 1, comprising Z-Nle-Lys-Arg-AMC.

6. A pharmaceutical or diagnostic composition comprising the peptidic inhibitor of cathepsin B of claim 1, and a pharmaceutically acceptable excipient.

7. A method of inhibiting neutral pH cathepsin B activity, comprising administering to a biological specimen an effective amount of a peptidic inhibitor of claim 1.

8. A method for treatment of a disease or disorder comprising administering to a mammalian subject in need an effective amount of a pharmaceutical composition comprising a peptidic inhibitor of claim 1.

9. The method of claim 8, wherein the disease or disorder is neurological.

10. The method of claim 8, wherein the disease is autoinflammatory disease, atherosclerosis, cancer, lung disease, pancreatitis, rheumatoid arthritis, viral infection, or TNF-alpha induced hepatic apoptotic cell death.

11. A method for detecting cathepsin B activity in a composition of interest comprising administering to the composition an effective amount of a composition comprising a peptidic inhibitor of claim 1, and detecting cathepsin B activity.
